(19) United States
(12) Patent Application Publication
(10) Pub. No.: US 2026/0232678 A1
(43) Pub. Date: Aug. 13, 2026

(54) Nasal Spray for Phosphodiesterase Inhibitors

(71) Applicant: Cyrano Therapeutics, Inc., Delray Beach, FL (US)

(72) Inventors: Richard GEOFFRION, Delray Beach, FL (US); Ronald KUPPERSMITH, College Station, TX (US)

(21) Appl. No.: 19/651,054

(22) Filed: Apr. 17, 2026

Related U.S. Application Data
(63) Continuation of application No. 18/760,124, filed on Jul. 1, 2024.
Application No. 18/760,124 is a continuation of application No. PCT/US2023/060164, filed on Jan. 5, 2023.
(60) Provisional application No. 63/297,060, filed on Jan. 6, 2022.
(60) Provisional application No. 63/418,661, filed on Oct. 24, 2022.

(51) Int. Cl. A61K 31/522; A61K 9/00; A61K 31/44; A61K 31/4515; A61K 31/4706; A61K 31/5415; A61K 31/5513; A61P 27/00

(52) U.S. Cl. CPC A61K 31/522; A61K 9/0043; A61K 31/44; A61K 31/4515; A61K 31/4706; A61K 31/5415; A61K 31/5513; A61P 27/00

(57) ABSTRACT

Disclosed herein are methods of treating a chemosensory dysfunction in a subject, comprising intranasally administering to the subject a phosphodiesterase inhibitor or a salt thereof by a nasal spray device. Also disclosed herein are devices and kits for administering phosphodiesterase inhibitors or salts thereof to a subject in need thereof. The nasal spray devices herein deliver a dosage unit that can be used to treat chemosensory dysfunction from a viral infection or another underlying cause of chemosensory dysfunction.

### CROSS-REFERENCE

This application is a continuation of U.S. patent application Ser. No. 18/760,124, filed Jul. 1, 2024, which is a continuation of PCT Application No. PCT/US2023/060164, filed Jan. 5, 2023, which claims the benefit of U.S. Provisional Application No. 63/297,060, filed Jan. 6, 2022, and of U.S. Provisional Application No. 63/418,661, filed Oct. 24, 2022, the disclosures of which are incorporated herein by reference in their entirety.

### SUMMARY

Disclosed herein are methods of treating chemosensory dysfunction in a subject in need thereof comprising administering to the subject an effective amount of a liquid pharmaceutical composition that comprises: a phosphodiesterase (PDE) inhibitor and a pharmaceutically acceptable carrier, excipient, diluent, or any combination thereof. In some embodiments, the liquid pharmaceutical composition, upon intranasal administration to the subject by actuation of a nasal spray device that comprises the liquid pharmaceutical composition, forms a plume comprising a plurality of droplets characterized by: a D₉₀ of from about 38 μm to about 49 μm, wherein about 90% of the droplets in the plume have a size less than the D₉₀. In some embodiments, the plurality of droplets can be further characterized by less than about 1.5% of the droplets in the plume having a size of less than about 10 μm. In some embodiments, the plurality of droplets can be further characterized by a D₅₀ of about 23 μm to about 30 μm, wherein about 50% of the droplets in the plume have a size less than the D₅₀. In some embodiments, the D₅₀ is about 25 μm. In some embodiments, the droplet size can be measured by a laser diffraction. In some embodiments, a PDE inhibitor or the salt thereof can comprise a theophylline or a salt thereof, a cilostazol or a salt thereof, a roflumilast or a salt thereof or any combination thereof. In some embodiments, a PDE inhibitor or the salt thereof can be theophylline or a salt thereof. In some embodiments, the plume can be formed by an actuation of the nasal spray device and can last about 0.5 seconds to about 5 seconds from the start of a spray to the end of the spray. In some embodiments, a pharmaceutically acceptable carrier can comprise a water. In some embodiments, the liquid pharmaceutical composition can further comprise a viscosity enhancer. In some embodiments, the viscosity enhancer can comprise a cellulose. In some embodiments, the liquid pharmaceutical composition can further comprise an excipient. In some embodiments, the excipient can comprise a glycerol. In some embodiments, the liquid pharmaceutical composition can further comprise a preservative. In some embodiments, the actuation can comprise an actuation amount of about 20 μl to about 80 μl of liquid. In some embodiments, intranasal administration can be once, twice, or thrice daily to each naris. In some embodiments, a plume can cover about 15% to about 50% of the surface area of a nasal cavity as measured by a nasal cast scan. In some embodiments, the nasal cavity can comprise a nasal septum, a nasal floor, a lateral nasal wall, an inferior meatus, a middle meatus, a superior meatus, an olfactory cleft, an olfactory region, a nasal turbinate, or any combination thereof. In some embodiments, the nasal cavity can comprise a nasal septum, a nasal floor, a lateral nasal wall, an inferior meatus, a middle meatus, a superior meatus, an olfactory cleft, an olfactory region, and a nasal turbinate. In some embodiments, the liquid pharmaceutical composition can be in a unit dose and can comprise about 20 μg to about 2000 μg of the PDE inhibitor or the salt thereof. In some embodiments, the liquid pharmaceutical composition is in a dosage unit comprising a PDE inhibitor or the salt thereof and can comprise dosage of about 20 μg to about 12,000 μg. In some embodiments, a total dose, which can, for example, be independently dosed once, twice, three time, four times, or five times a day, in a single naris or divided up between two nares, of a PDE inhibitor or a salt thereof, can be delivered by one or more actuations of an intranasal delivery device and can range be from about 1 microgram to about 100 mg, or from about 1 microgram to about 50 mg, or from about 1 microgram to about 20 mg, or from about 1 microgram to about 10 mg, or from about 1 microgram to about 9 mg, or from about 1 microgram to about 8 mg, or from about 1 microgram to about 7 mg, or from about 1 microgram to about 6 mg, or from about 1 microgram to about 5 mg, or from about 1 microgram to about 4 mg, or from about 1 microgram to about 3 mg, or from about 1 microgram to about 2 mg, or from about 1 microgram to about 1 mg, or from 1 microgram to about 0.5 mg, or from about 10 micrograms to about 2 mg, or from about 20 micrograms to about 2 mg, or from about 40 micrograms to about 2 mg, or from about 60 micrograms to about 2 mg, or from about 80 micrograms to about 2 mg, or from about 100 micrograms to about 2 mg, or from about 200 micrograms to about 2 mg, or from about 400 micrograms to about 2 mg, or from about 600 micrograms to about 2 mg, or from about 800 micrograms to about 2 mg, or from about 1000 micrograms to about 2 mg. The total dose can be contained in a liquid pharmaceutical composition. The total dose of the PDE inhibitor or the salt thereof, which can be contained in liquid pharmaceutical compositions, can be administered once, twice, three, four, five, or six times daily, and can be administered in on naris, or split between two nares. The total dose of the PDE inhibitor of the salt thereof can be, for example, about: 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000 micrograms, and this can be contained in a liquid pharmaceutical composition. The total dose of the PDE inhibitor of the salt thereof can be delivered by administering 1 or more unit doses of containing the PDE inhibitor or the salt thereof, which can be contained in a liquid pharmaceutical composition, from an intranasal delivery device, delivered into one naris or two nares, for example about: 1, 2, 3, 4, 5, 6, 7, 8, 9, 10, 11, 12, 13, 14, 15, 16, 17, 18, 19, 20, 21, 22, 23, 24, 25, 26, 27, 28, 29 30, 31 32, 33, 34, 35, 36, 37, 38, 39, 40, 41, 42, 43, 44, 45, 46, 47, 48, 49, or 50 unit doses. A unit dose can independently contain the PDE inhibitor or the salt there of an amount of about: 10, 20 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 2000, 3000, 4000, 5000, 6000, 7000, 8000, 9000, 10,000, 20,000, 30,000, 40,000, 50,000, 60,000, 70,000, 80,000, 90,000, 100,000, 110,000, or 120,000 micrograms; and the unit dose can be of a liquid pharmaceutical composition. In some embodiments, chemosensory dysfunction can be ageusia, hypogeusia, dysgeusia, parosmia, phantosmia, anosmia, hyposmia, dysosmia, or any combination thereof. In some embodiments, the chemosensory dysfunction can be anosmia, hyposmia, dysosmia, parosmia, or a combination thereof. In some embodiments, the chemosensory dysfunction can be smell loss, taste loss, or a combination thereof. In some embodiments, the chemosensory dysfunction can arise after a viral infection. In some embodiments, the viral infection can comprise a coronavirus infection or an influenza infection. In some embodiments, the subject prior to the administering, can have a decreased level of a cyclic nucleotide in a nasal mucus sample from the subject in comparison to a cyclic nucleotide level of a control population with normal chemosensory function. In some embodiments, the subject prior to the administering, can have a decreased level of sonic hedgehog in a nasal mucus sample from the subject in comparison to a sonic hedgehog level of a control population with normal chemosensory function. In some embodiments, the method can further comprise administering a second therapeutic. In some embodiments, the second therapeutic can be administered concurrently or consecutively with the administering. In some embodiments, the method can further comprise diagnosing the subject with chemosensory dysfunction. In some embodiments, the subject was previously diagnosed with chemosensory dysfunction. In some embodiments, the nasal spray device upon actuation can deliver a plume as a unit dose. In some embodiments, the nasal spray device comprises about 60 to about 120 unit doses. In some embodiments, each unit dose comprises about 20 μg to about 2000 μg of the PDE inhibitor or the salt thereof. In some embodiments, the subject is a subject in need thereof. In some embodiments, the subject is a human. In some embodiments, upon actuation, the nasal spray device can deliver about 35 mg to about 50 mg of the pharmaceutical composition. In some embodiments, the plurality of droplets can be further characterized by a D₁₀ of about 14 μm to about 19 μm, wherein about 10% of the droplets in the plume have a size less than the D₁₀. In some embodiments, a stroke length of the actuation of the nasal spray device can be 4.6 mm, 4.8 mm, or 4.9 mm. In some embodiments, an actuator stroke velocity of the actuation of the nasal spray device can be 2 mm/s or 3 mm/s. In some embodiments, an actuator stroke acceleration of the actuation of the nasal spray device can be about 500 mm/s². In some embodiments, a nasal spray device can have a nozzle pore size of about 3 μm, about 4 μm, or about 5 μm. In some embodiments, a nasal spray device can have about 40 to about 70 nozzle pores, or about 45 pores to about 65 nozzle pores. In some embodiments, the nasal spray device can have a cone angle of about 20 degrees.

Also disclosed herein are nasal spray devices comprising: a phosphodiesterase (PDE) inhibitor. In some embodiments, a nasal spray device can be configured to deliver a dosage unit in a plume upon actuation. In some embodiments, a dosage unit can comprise a therapeutically effective amount of the PDE inhibitor in a pharmaceutically acceptable carrier comprising an excipient. In some embodiments, a plume can have a droplet size distribution characterized by: (a) less than about 1.5% of the droplets in the plume having a size of less than about 10 μm; and (b) a D₉₀ of from about 38 μm to about 49 μm, wherein about 90% of the droplets in the plume have a size less than the D₉₀. Also disclosed herein are kits comprising a nasal spray device and a container.

Also disclosed herein are methods of treating a chemosensory dysfunction in a subject in need thereof comprising administering a phosphodiesterase (PDE) inhibitor using a nasal spray device that delivers a dosage unit in a plume upon an actuation that comprises a therapeutically effective amount of a PDE inhibitor in a liquid pharmaceutical composition comprising a pharmaceutically acceptable carrier, diluent, excipient, or any combination thereof, wherein the plume comprises droplets and has a droplet size distribution characterized by: (a) less than about 1.5% of the droplets in the plume having a size of less than about 10 μm; and (b) a D₉₀ of from about 38 μm to about 49 μm, wherein about 90% of the droplets in the plume have a size less than the D₉₀.

Also disclosed herein are methods of treating a chemosensory dysfunction in a subject in need thereof comprising administering to the subject an effective amount of a liquid pharmaceutical composition that comprises: a) thioridazine, haloperidol, olanzapine, a salt of any of these, or any combination thereof; and b) a pharmaceutically acceptable carrier, excipient, diluent, or any combination thereof. In some embodiments, the liquid pharmaceutical composition, upon intranasal administration to the subject by actuation of a nasal spray device that comprises the liquid pharmaceutical composition, can form a plume comprising a plurality of droplets characterized by: a D₉₀ of from about 38 μm to about 49 μm, wherein about 90% of the droplets in the plume have a size less than the D₉₀. In some embodiments, the administering of the effective amount of the liquid pharmaceutical composition treats the chemosensory dysfunction.

In some embodiments, the chemosensory dysfunction is phantosmia, phantogeusia, or both.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows images of a nasal cavity model after being sprayed with a soft mist nasal spray device, a slow standard nasal spray device, and a standard nasal spray device. The liquid formulation in the spray devices was tested with and without a cellulose (i.e., carboxymethylcellulose) viscosity enhancer. The soft mist nasal spray device deposited a higher amount of the formulation in the olfactory region of the nasal cavity model.

FIG. 2 shows the nasal spray characteristics of three spray devices: a soft mist nasal spray device, a slow standard nasal spray device, and a standard nasal spray device. The soft mist nasal spray device displayed a spray distribution wherein about 50% of the particles were less than about 20 μm in diameter as compared to the standard nasal sprays (i.e., the standard nasal spray and the slow standard nasal spray) which had sprays comprising larger particle diameters.

FIG. 3 depicts a box and whisker graph showing the metered shot weight (amount released in milligrams (mg)) from soft mist pump devices at different actuation velocities (1, 2, 3, and 4 millimeters per second (mm/s)) and at 4.6 mm, 4.8 mm, and 4.9 mm stroke lengths.

FIG. 4 depicts a box and whisker graph showing the delivered shot weight (amount delivered in mg) from soft mist pump devices at different actuation velocities (1, 2, 3, and 4 millimeters per second (mm/s)) and at 4.6 mm, 4.8 mm, and 4.9 mm stroke lengths.

FIG. 5 depicts a box and whisker graph showing the plume geometry performance (Plume Angle (degree) and Plume Width (mm)) at a 60 mm pattern distance from soft mist pump devices at different actuation velocities (1, 2, 3 millimeters per second (mm/s)) and at a 4.8 mm stroke length.

FIG. 6 depicts a box and whisker graph showing the spray pattern (Dmax (mm), Dmin (mm), ovality and area (mm\hat{ }2)) at 30 mm and 60 mm pattern distances from soft mist pump devices at different actuation velocities (2 and 3 mm/s) and at a 4.8 mm stroke length.

FIG. 7 depicts a images showing the spray patterns at 30 mm and 60 mm pattern distances from a soft mist pump device (device 12) at different actuation velocities (2 and 3 millimeters per second (mm/s)) and at a 4.8 mm stroke length.

FIG. 8 depicts a box and whisker graph showing the droplet size distribution (% volume <10 μm; span; D₉₀ value (μm); and D₅₀ value (μm)) at 30 mm and 60 mm pattern distances from soft mist pump devices at different actuation velocities (2 and 3 mm/s) and at a 4.8 mm stroke length.

FIG. 9 depicts a box and whisker graph showing the plume geometry performance (Plume Angle (degree) and Plume Width (mm)) at a 60 mm pattern distance from soft mist pump devices at different actuation velocities (1, 2, 3 millimeters per second (mm/s)) and at a 4.6 and 4.8 mm stroke length.

FIG. 10 depicts a box and whisker graph showing the spray pattern (Dmax (mm), Dmin (mm), ovality and area (mm\hat{ }2)) at a 30 mm pattern distance from soft mist pump devices at different actuation velocities (2 and 3 mm/s) and at 4.6 mm and 4.8 mm stroke lengths.

FIG. 11 depicts a box and whisker graph showing the spray pattern (Dmax (mm), Dmin (mm), ovality and area (mm\hat{ }2)) at a 60 mm pattern distance from soft mist pump devices at different actuation velocities (2 and 3 mm/s) and at 4.6 mm and 4.8 mm stroke lengths.

FIG. 12 depicts a box and whisker graph showing the droplet size distribution (% volume <10 μm; span; D₉₀ value (μm); and D₅₀ value (μm)) at 30 mm pattern distances from soft mist pump devices at different actuation velocities (2 and 3 mm/s) and at 4.6 mm and 4.8 mm stroke lengths.

FIG. 13 depicts a box and whisker graph showing the droplet size distribution (% volume<10 μm; span; D₉₀ value (μm); and D₅₀ value (μm)) at 60 mm pattern distances from soft mist pump devices at different actuation velocities (2 and 3 mm/s) and at 4.6 mm and 4.8 mm stroke lengths.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The singular forms “a”, “an”, and “the” are used herein to include plural references unless the context clearly dictates otherwise. Accordingly, unless the contrary is indicated, the numerical parameters set forth in this application are approximations that can vary depending upon the desired properties sought to be obtained.

The terms “determining”, “measuring”, “evaluating”, “assessing,” “assaying,” and “analyzing” are often used interchangeably herein to refer to forms of measurement and include determining if an element may be present or not (for example, detection). These terms can include quantitative and qualitative determinations. Assessing can be alternatively relative or absolute. “Detecting the presence of” includes determining the amount of something present, as well as determining whether it may be present or absent.

The term “substantially” or “essentially” refers to a qualitative condition that exhibits an entire or nearly total range or degree of a feature or characteristic of interest. In some cases, substantially refers to at least about: 70%, 75%, 80%, 85%, 90%, 95%, 99%, 99.9% or 99.99% of the total range or degree of a feature or characteristic of interest. In some cases, the substantially or essentially refers to an amount that can be about 100% of a total amount.

The term “at least partially” refers to a qualitative condition that exhibits a partial range or degree of a feature or characteristic of interest. In some cases, at least partially refers to at least about: 5%, 10%, 20%, 30%, 40%, 50%, 60%, 70%, 75%, 80%, 85%, 90%, 95%, 99%, or 100% of the total range or degree of a feature or characteristic of interest.

Unless otherwise indicated, open terms for example “contain,” “containing,” “include,” “including,” and the like mean comprising.

As used herein, the term “about” or “approximately” means within an acceptable error range for the particular value as determined by one of ordinary skill in the art, which will depend in part on how the value is measured or determined, e.g., the limitations of the measurement system. For example, “about” means plus or minus 10%, per the practice in the art. Alternatively, “about” means a range of plus or minus 20%, plus or minus 10%, plus or minus 5%, or plus or minus 1% of a given value. Alternatively, particularly with respect to biological systems or processes, the term means within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

When used herein, a percentage of a compound of a composition is with respect to a total weight or a total volume of a composition. In some cases, a percentage of a component of a composition is with respect to a total weight or a total volume of a composition

The terms “administer,” “administering”, “administration,” and the like, as used herein, refers to methods that are used to enable delivery of compounds or their salts or compositions to the desired site of biological action. Delivery can include direct application to the affect tissue or region of the body. A composition provided herein can be administered by any method. A method of administration is by a nasal spray. In some cases, a method of administration can be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, nasal administration, inhalation administration, intraduodenal administration, rectal administration. Delivery can include topical administration (such as a lotion, a cream, a gel, a liquid, a solid, a powder, an ointment) to an external surface of a surface, such as a skin. In some instances, a subject administers an intranasal spray comprising the compound in the absence of supervision. In some instances, a subject administers the intranasal formulation under the supervision of a medical professional (e.g., a physician, nurse, physician's assistant, orderly, hospice worker, etc.). In some cases, a medical professional administers the intranasal formulation. In some cases, a cosmetic professional administers the intranasal formulation.

As used herein, “treating” of chemosensory dysfunction includes one or more of: reducing the frequency or severity of one or more symptoms, prevention of one or more symptoms or their underlying cause, elimination of one or more symptoms or their underlying cause, or improvement or remediation of damage. For example, treatment of chemosensory dysfunction can include, increasing smell acuity and taste acuity from a patient suffering from a viral infection, such as a patient with COVID-19 or influenza and/or causing the regression or disappearance of chemosensory dysfunction.

A “therapeutically effective amount” refers to an amount of a compound or its salt with or without additional agents that is effective to achieve its intended purpose. Individual patient needs may vary. Generally, the dosage required to provide an effective amount of the compound, salt thereof, or composition containing one or both of these will vary, depending on the age, health, physical condition, sex, weight, extent of the dysfunction of the recipient, frequency of treatment and the nature and scope of the dysfunction.

A “dosage unit” as used herein, refers to a discrete amount of a pharmaceutical composition that is administered in a single event or package. The meaning of the term dosage unit is context specific. For example, a dosage unit for an intranasally administered liquid pharmaceutical composition would be the volume of the composition that is administered in a single event. For a nasal spray, the dosage unit would be the volume of the composition that is released upon each actuation of the nasal spray device.

“Percentage by weight” or “w/w” means ratio of the mass of the specified ingredient verses the mass of the entire composition (e.g., dosage unit).

“Plume geometry” or “geometry” when used in connection with a plume, means the measurement of the angle of the plume at its origin. Plume geometry can be measured at two distances from the origin of the plume, for example, at two side views 90° relative to each other. Plume geometry can also be calculated from the spray pattern.

“Spray pattern,” “plume ovality,” or “ovality” when used in connection with a plume, refers to shape and size of the plume at a distance from its origin. “Ovality” can be measured as the ratio of the largest diameter to the smallest diameter.

“D₁₀,” “D₅₀,” “D₉₀,” and “span” are measurements of the droplet or particle size distribution of a plume. In a plume, 10% of the droplets have a size less than the D₁₀, 50% of the droplets have a size less than the D₅₀, and 90% of the droplets have a size less than the D₉₀. The span can be calculated from these numbers according to the following formula: span=(D₉₀−D₁₀)/D₅₀. In some cases, a D₁₀, D₅₀, or D₉₀ value can be a mean, or a median value from a plurality of sprays and/or droplets.

“Total volume,” when used in connection with a plume, refers to the total volume of all droplets or particles in the plume. For example, a plume with a total volume of 100 μL contains 100 μL of liquid.

The term “subject,” “host,” “individual,” and “patient” are as used interchangeably herein to refer to animals, typically mammalian animals. Any suitable mammal can be administered a compound, salt, or a composition as described herein or be treated by a method as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals can be any age or at any stage of development, for example a mammal can be neonatal, infant, adolescent, adult or in utero. In some embodiments a mammal is a human. Humans can be more than about: 1, 2, 5, 10, 20, 30, 40, 50, 60, 65, 70, 75, 80, 85, 90, 95, 100, 105, 110, 115 or about 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, a human can be less than about 18 years of age. In some cases, human can be from about 1 month to about 12 months old, from about 1 year to about 20 years, from about 15 years to about 50 years, from about 40 years to about 80 years, or from about 60 years to about 110 years. In some cases, a human can be more than about 18 years of age. A mammal such as a human can be male or female. In some embodiments, a subject can have or can be suspected of having a disease or condition. The subject can be a patient, such as a patient being treated for a condition or a disease, such as a heart disease, hypertension, atrial fibrillation, stroke, renal failure, liver disease, cancer, diabetes, respiratory disease, asthma, chronic obstructive pulmonary disease, bronchitis, emphysema, lung cancer, cystic fibrosis, a Coronavirus infection, an influenza infection, a viral infection, a bacterial infection, a fungal infection, a parasitic infection, pneumonia, pleural effusion, or any combination thereof. A subject can be predisposed to a risk of developing a condition or a disease such as a respiratory disease. A subject can be in remission from a condition or a disease, such as a cancer patient. In some instances, a subject can be healthy.

As disclosed herein, the term “phosphodiesterase (PDE) inhibitor” refers to a compound or its salt that can at least partially inhibit the function of a phosphodiesterase (PDE) polypeptide, such as a PDE1, a PDE2, a PDE3, a PDE4, a PDE5, a PDE6, a PDE7, a PDE8, a PDE9, a PDE10, a PDE11 polypeptide, or any combination thereof.

As used herein, reference to a PDE inhibitor generally, or a specific PDE inhibitor, includes reference to any salt, solvate, ester, or polymorph of the PDE inhibitor. A “salt” can include a pharmaceutically acceptable salt. Examples of pharmaceutically acceptable salts can include those salts prepared by reaction of a compound disclosed herein with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate. Further, a compound disclosed herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. Other acids, such as oxalic, while not in themselves pharmaceutically acceptable, can be employed in the preparation of salts useful as intermediates in obtaining a compound and/or a pharmaceutically acceptable acid addition salt. In some embodiments, a compound disclosed herein which can comprise a free acid group reacts with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts can include the lithium, sodium, potassium, calcium, magnesium, and aluminum salts and the like. Illustrative examples of bases can include sodium hydroxide, potassium hydroxide, choline hydroxide, sodium carbonate, N+(C1-4 alkyl)4, and the like. Representative organic amines useful for the formation of base addition salts can include ethylamine, diethylamine, ethylenediamine, ethanolamine, diethanolamine, piperazine and the like. It may be understood that a compound disclosed herein can also include the quaternization of any basic nitrogen-containing groups they contain. In some embodiments, water or oil-soluble or dispersible products can be obtained by such quaternization. A compound disclosed herein can be prepared as pharmaceutically acceptable salts formed when an acidic proton present in the parent compound either can be replaced by a metal ion, for example an alkali metal ion, an alkaline earth ion, or an aluminum ion; or coordinates with an organic base. In some embodiments, base addition salts can be also prepared by reacting the free acid form of a compound disclosed herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

The section headings used herein are for organizational purposes only and are not to be construed as limiting the subject matter described.

### Overview

Disclosed herein are methods, compositions, kits, and devices for the treatment of chemosensory dysfunction with phosphodiesterase inhibitors. In some embodiments, methods of treatment can comprise administering to a subject a phosphodiesterase (PDE) inhibitor, its salt, or a combination of PDE inhibitors or their salts by a nasal spray device. The nasal spray device can release a dosage unit in a plume upon actuation. In some embodiments, the nasal spray device has been configured to release a plume with a D₅₀ value of about 20 μm. In some cases, a PDE inhibitor can be administered as an intranasal formulation. In some cases, a PDE inhibitor can inhibit a PDE polypeptide, and can comprise a theophylline or a salt thereof, a cilostazol or a salt thereof, a roflumilast or a salt thereof or any combination thereof.

### Phosphodiesterase (PDE) Polypeptides and Inhibitors

Disclosed herein are PDE inhibitors for the treatment of chemosensory dysfunction. PDE inhibitors can be selective or nonselective to different phosphodiesterase enzymes. The compositions disclosed herein, such as nasal sprays comprise phosphodiesterase inhibitors or salts thereof and are administered to subjects in need thereof.

PDE1 (phosphodiesterase type 1) is an enzyme known as calcium- and calmodulin-dependent phosphodiesterase. Different tissues such as the heart, lung and brain express PDE1. PDE1 can hydrolyze both ribonucleotides an deoxyribonucleotides. PDE1 enzymes can degrade both cGMP and cAMP. PDE1 enzymes can play a role in smooth muscle proliferation and cell signaling pathways.

PDE2 polypeptides can decrease aldosterone secretion. Such decrease may play an important role in the regulation of elevated intracellular concentrations of cAMP and cGMP in platelets. Several regions of the brain can express PDE2 and rat experiments indicate that inhibition of PDE2 can enhance memory. PDE2 may play a role in regulation of fluid and cell extravasation during inflammatory conditions as PDE2 can be localized to microvessels, especially venous capillary and endothelial cells. PDE2 may also be a good pharmacological target for pathological states such as sepsis or in more localized inflammatory responses such as thrombin-induced edema formation in the lung.

The PDE3 family hydrolyzes cAMP and cGMP, but in a manner suggesting that in vivo, the hydrolysis of cAMP can be inhibited by cGMP. They can also be distinguished by their ability to be activated by several phosphorylation pathways including the PKA and PI3K/PKB pathways. PDE3A can be relatively highly expressed in platelets, as well as in cardiac myocytes and oocytes. PDE3B can be a major PDE in adipose tissue, liver, and pancreas, as well as in several cardiovascular tissues. Both PDE3A and PDE3B can be highly expressed in vascular smooth muscle cells and are likely to modulate contraction. PDE4 polypeptides can regulate the production of pro-inflammatory and anti-inflammatory cytokines, and cell proliferation via the degradation of cAMP. PDE5 can be a regulator of vascular smooth muscle contraction best known as the molecular target for several well-advertised drugs used to treat erectile dysfunction and pulmonary hypertension. In the lung, inhibition of PDE5 can oppose smooth muscle vasoconstriction, and PDE5 inhibitors are in clinical trials for treatment of pulmonary hypertension.

Examples of a PDE inhibitor can include, for example, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI-930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, aminone, tolafentrine, dipyridamole, papaverine, E4021, thienopyrimidine derivatives, triflusal, ICOS-351, tetrahydropiperazino[1,2-b]beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, salts of any of these, and the like.

A PDE inhibitor can be a selective PDE inhibitor, or a non-specific PDE inhibitor. A PDE selective inhibitor can include a PDE1 selective inhibitor, PDE2 selective inhibitor, PDE3 selective inhibitor, PDE4 selective inhibitor, PDE5 selective inhibitor, a PDE6 selective inhibitor, a PDE7 selective inhibitor, a PDE8 selective inhibitor, a PDE9 selective inhibitor, a PDE10 selective inhibitor, or a PDE11 selective inhibitor. In some cases, a selective PDE inhibitor can be specific for more than one of PDE1, PDE2, PDE3, PDE4, PDE5, PDE6, PDE7, PDE8, PDE9, PDE10, and PDE11. A non-specific PDE can include a PDE inhibitor that inhibits at least two, three, four, or five or more of PDE1, PDE2, PDE3, PDE4, PDE, PDE6, PDE7, PDE8, PDE9, PDE10, and PDE11.

A PDE inhibitor can inhibit cellular apoptosis by inhibiting TNF alpha, TRAIL and their metabolites. PDE inhibitors can activate the production and secretion of nitric oxide in all tissues thereby inducing vasorelaxation or vasodilation of all blood vessels including those of the peripheral blood vessels (inhibiting intermittent claudication), the distal extremities and in the penile region contributing to penile erection.

A non-specific PDE inhibitor can include theophylline, papaverine, caffeine, IBMX (3-isobutyl-1-methylxanthine, aminophylline, doxophylline, cipamphylline, theobromine, pentoxifylline (oxpentifylline), diprophylline, or a salt of any of these. Theophylline is a methylxanthine derivative that, when administered as described herein, can be used to treat chemosensory dysfunction. In some cases, an anti-inflammatory effect can be achieved when theophylline is prescribed at or administered at levels that produce systemic levels of theophylline in the blood well below that which causes side effects. Patients with emphysema and chronic bronchitis can also be helped with theophylline when their symptoms are partially related to reversible airway narrowing.

A PDE1 selective inhibitor, formerly known as calcium- and calmodulin-dependent phosphodiesterases, can include ebumamenine-14-carboxylic acid ethyl ester (vinpocetine). In some cases, vinpocetine can be used to induce vasorelaxtion on cerebral smooth muscle tissue. In some cases, a PDE1 selective inhibitor can comprise IC86340, Amiodarone, Lisinopril, 8-methoxymethyl-IBMX, a salt of any of these, or any combination thereof. In some cases, a PDE1 selective inhibitor can comprise vinpocetine or a salt thereof.

A PDE2 selective inhibitor can include EHNA (erythro-9-(2-hydroxy-3-nonyl) adenine), 9-(6-phenyl-2-oxohex-3-yl)-2-(3,4-dimethoxybenzyl)-purin-6-one (PDP), BAY 60-7750, or a salt of any of these.

A PDE3 selective inhibitor can include enoximone, milrinone (Primacor), amrinone, cilostamide, cilostazol (Pletal), trequinsin, or a salt of any of these. A PDE3 inhibitor when administered as described herein can produce sympathetic stimulation to increase cardiac inotropy, chronotropy and dromotropy. A PDE3 inhibitor when administered as described herein can also antagonize platelet aggregation, increase myocardial contractility, and enhance vascular and airway smooth muscle relaxation. PDE3A can be a regulator of this process. A PDE3 inhibitor when administered as described herein can effectively prevent aggregation. Cilastazol (Pletal), is approved for treatment of intermittent claudication. Its mechanism of action may involve inhibition of platelet aggregation along with inhibition of smooth muscle proliferation and vasodilation.

A PDE4 selective inhibitor can include mesembrine, rolipram, Ibudilast (i.e., a neuroprotective and bronchodilator drug that can be used in the treatment of asthma and stroke), and roflumilast (Daxas), cilomilast (Airflo), or a salt of any of these. In some cases, a PDE4 selective inhibitor can be administered for treatment of chronic obstructive pulmonary disease. A PDE4 selective inhibitor can at least partially suppress release of inflammatory mediators e.g., cytokines, or at least partially inhibit production of reactive oxygen species and immune cell infiltration. A PDE4 inhibitor can also be used to treat asthma, arthritis, and psoriasis.

A PDE5 selective inhibitor can include sildenafil, tadalafil, vardenafil, udenafil, avanafil and the salts thereof.

### Additional Therapeutics

In some embodiments, a composition herein can comprise an anticholinergic. In some embodiments, an anticholinergic can comprise thioridazine, haloperidol, olanzapine, a salt of any of these or any combination thereof. In some cases, an anticholinergic can comprise an amitriptyline, an atropine, an aclidinium, a benztropine, a chlorpheniramine, a chlorpromazine, a clomipramine, a clozapine, a cyclobenzaprine, a cyproheptadine, a darifenacin, a desipramine, a dexchlorpheniramine, a dicyclomine, a diphenhydramine, a doxepin, a hydroxyzine, a hyoscyamine, an imipramine, a meclizine, a nortriptyline, an olanzapine, an orphenadrine, an oxybutynin, a paroxetine, a perphenazine, a prochlorperazine, a promethazine, a protriptyline, a pseudoephedrine hcl/triprolidine hcl, a scopolamine, a thioridazine, a tolterodine, a trifluoperazine, a trihexyphenidyl, a trimipramine, a salt of any of these, or any combination thereof.

In some embodiments, a composition can comprise a luteolin, a salt thereof, or a derivative thereof. In some cases, a composition can comprise a palmitoylethanolamide, a derivative thereof, or a salt thereof. In some cases, a composition can comprise a palmitoylethanolamide, a derivative thereof, or a salt thereof and a luteolin, a derivative thereof, or a salt thereof. In some cases, a palmitoylethanolamide, a derivative thereof, or a salt thereof and/or a luteolin a derivative thereof, or a salt thereof can comprise an active agent in a composition, such as a pharmaceutical composition. In some cases, a palmitoylethanolamide, a derivative thereof, a salt thereof, a luteolin, a derivative thereof, or a salt thereof can be mixed with a PDE inhibitor or salt thereof. In some cases, a palmitoylethanolamide, a derivative thereof, a salt thereof, a luteolin, a derivative thereof, a salt thereof, or any combination thereof can be administered by a spray device. In some cases, the PDE inhibitor can comprise theophylline or a salt thereof. In some cases, a palmitoylethanolamide, a derivative thereof, or a salt thereof, and a luteolin, a derivative thereof, or a salt thereof can be mixed with a PDE inhibitor or salt thereof. In some cases, a palmitoylethanolamide, a derivative thereof, or a salt thereof and/or a luteolin a derivative thereof, or a salt thereof can be used to treat a chemosensory dysfunction. In some instances, a palmitoylethanolamide, a derivative thereof, a salt thereof, a luteolin, a derivative thereof, a salt thereof, a PDE inhibitor or a salt thereof, can each independently be present in a therapeutically effective amount to treat a chemosensory dysfunction. In some cases, a PDE inhibitor or a salt thereof can be administered intranasally and a palmitoylethanolamide, a derivative thereof, a salt thereof, a luteolin, a derivative thereof, a salt thereof, or any combination thereof can be administered orally, for example in the form of a pill, a liquid, a capsule or a tablet.

In some embodiments representative dosages of a palmitoylethanolamide, a derivative thereof, a salt thereof, a luteolin, a derivative thereof, or a salt thereof can be from about 1.0 μg to 2000 mg per day, from about 1.0 μg to 500.0 mg per day, from about 10 μg to 100.0 mg per day, from about 10 g to about 10 mg per day, from about 10 g to 1.0 mg per day, from about 10 μg to 500 μg per day, from about 20 μg to about 2000 μg per day, from about 100 μg to about 10,000 μg per day, or from about 1 μg to 50 μg per day. These ranges of dosage amounts represent total dosage amounts of the active ingredient per day for a given patient. In some embodiments, a daily administered dose can be less than about, or equal to: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, a daily administered dose can be at least about: 2000 mg per day, 1000 mg per day, 500 mg per day, 100 mg per day, 10 mg per day, 1.0 mg per day, 500 g per day, 300 g per day, 200 μg per day, 100 μg per day or 50 μg per day. In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day.

### Compositions and Formulations

In some embodiments, a composition or a formulation described herein can comprise an active ingredient, a carrier, an excipient, a viscosity enhancer, a diluent, a preservative, an enzyme modulator, a vasoconstrictor, a mucoadhesive, a humectant, or any combination thereof. In some cases, a carrier, an excipient, a viscosity enhancer, a diluent, a preservative, a humectant, an enzyme modulator, a vasoconstrictor, or a mucoadhesive can be pharmaceutically acceptable versions thereof. In some embodiments, a composition or a formulation described herein can be a liquid. In some cases, an active ingredient can comprise a PDE inhibitor as described herein. For example, a PDE inhibitor can comprise theophylline or a salt thereof, a cilostazol or a salt thereof, a roflumilast or a salt thereof or any combination thereof. In some cases, a carrier can comprise a water. In some cases, a viscosity enhance can comprise cellulose. In some cases, an excipient can comprise glycerol. In some cases, a composition or a formulation described herein can be delivered by a nasal spray device. In some cases, a composition herein can be sterile. In some cases, a composition herein can be aseptic.

In some cases, a formulation or a composition can comprise an active agent such as a PDE inhibitor. In some cases, a composition or formulation can comprise an active agent in an amount of more than about: 0.01%, 0.02%, 0.03%, 0.04%, 0.05%, 0.06%, 0.07%, 0.08%, 0.09%, 0.1%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% (wt/wt) of the total composition. In some cases, a composition or formulation can comprise an active agent in an amount of less than about: 0.01%, 0.02%, 0.03%, 0.04%, 0.05%, 0.06%, 0.07%, 0.08%, 0.09%, 0.1%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, or 10% (wt/wt) of the total composition. In some cases, a composition or formulation can comprise an active agent in an amount of about: 0.01%, 0.02%, 0.03%, 0.04%, 0.05%, 0.06%, 0.07%, 0.08%, 0.09%, 0.1%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4% 5% 6%, 7%, 8%, 9%, or 10% (wt/wt) of the total composition. In some cases, a composition or formulation can comprise an active agent in an amount of about 0.01% to about 10% (wt/wt) of the total composition. In some cases, a composition or formulation can comprise one active agent. In some cases, a composition or formulation can comprise more than one active agent, for example a composition can comprise 2, 3, 4, 5, or more active agents.

In some embodiments, a composition or formulation can include an excipient. Excipients can include, but are not limited to: a water, a fluidizer, a lubricant, an adhesion agent, a surfactant, an acidifying agent, an alkalizing agent, an agent to adjust pH, an antimicrobial preservative, an antioxidant, an anti-static agent, a buffering agent, a chelating agent, a humectant, or a wetting agent. Excipients can also include a coloring agent, a coating agent, a sweetening agent, a flavoring and perfuming agent or a masking agent. A composition and formulation can include a therapeutic agent with an individual excipient or with multiple excipients in any suitable combination, with or without a carrier. In some cases, an excipient can comprise glycerol.

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, light mineral oil, lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a composition or formulation can comprise a viscosity enhancer (e.g., a solution thickener). In some instances, a viscosity enhancer can comprise a cellulose. In some cases, a viscosity enhancer can comprise a carboxymethylcellulose (CMC), a cellulose I, a cellulose II, a cellulose III, a cellulose IV, microcrystalline cellulose (MCC), sodium carboxymethyl cellulose (Na CMC), or a combination thereof. In some cases, a viscosity enhancer can comprise, cellulose acetate, cellulose triacetate, cellulose propionate, cellulose acetate propionate, cellulose acetate butyrate methylcellulose, ethyl cellulose, hydroxyethyl cellulose, hydroxypropyl cellulose (HPC), hydroxyethyl methyl cellulose, ethyl hydroxyethyl cellulose, hydroxypropyl methyl cellulose, or a combination thereof. In some cases, a composition or formulation can comprise a viscosity enhancer in an amount of about: 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9% or 10% (wt/wt) of the total composition. In some cases, a composition or formulation can comprise a viscosity enhancer in an amount of about 0.01% to about 10% (wt/wt) of the total composition. In some cases, a composition or formulation can comprise one viscosity enhancer. In some cases, a composition or formulation can comprise more than one viscosity enhancer, for example a composition can comprise 2, 3, 4, 5, or more viscosity enhancers.

In some cases, the viscosity of an intranasal dosage unit can affect the residence time in the nasal cavity. In some embodiments, the dosage unit has a higher kinematic viscosity than water at the same temperature. In some embodiments, the dosage unit can have a kinematic viscosity of from 0.5 cSt to 3 cSt at 20° C. For example, the dosage unit can have a kinematic viscosity of 0.5-2 cSt, 0.5-1.5 cSt, 0.5-1.25 cSt, 0.5-1.1 cSt, 0.5-1 cSt, 0.5-0.9 cSt, 0.5-0.75 cSt, 0.75-2 cSt, 0.75-1.5 cSt, 0.75-1.25 cSt, 0.75-1.1 cSt, 0.75-1 cSt, 0.75-0.9 cSt, 0.9-2 cSt, 0.9-1.5 cSt, 0.9-1.25 cSt, 0.9-1.1 cSt, 0.9-1 cSt, 1-2 cSt, 1-1.5 cSt, 1-1.25 cSt, 1-1.1 cSt, 1.1-2 cSt, 1.1-1.5 cSt, 1.1-1.25 cSt, 1.25-2 cSt, 1.25-1.5 cSt, 1.5-2 cSt, or 2-3 cSt at 20° C. In some embodiments, the dosage unit has a kinematic viscosity of 0.9-1.25 cSt at 20° C. In some embodiments, the dosage unit has a higher kinematic viscosity than water at the same temperature.

In some embodiments, a composition or formulation can comprise a carrier. In some cases, a carrier can comprise a water such as a purified water. In some cases, a carrier for a composition or formulation include but are not limited to amino acids, peptides, proteins, non-biological polymers, biological polymers, simple sugars, carbohydrates, gums, inorganic salts and metal compounds which may be present singularly or in combination. In some embodiments, a pharmaceutically acceptable carrier can comprise native, derivatized, modified forms, or combinations thereof. In some cases, proteins can include, but are not limited to, gelatin or albumin. In some embodiments, sugars that can serve as carriers include, but are not limited to fructose, galactose, glucose, lactitol, lactose, maltitol, maltose, mannitol, melezitose, myoinositol, palatinite, raffinose, stachyose, sucrose, trehalose, xylitol, hydrates thereof, and combinations of thereof. In some cases, carbohydrates that can serve as carriers include, but are not limited to starches such as corn starch, potato starch, amylose, amylopectin, pectin, hydroxypropyl starch, carboxymethyl starch, and cross-linked starch. In other embodiments, useful carbohydrates that can serve as pharmaceutically acceptable carriers include, but are not limited to cellulose, crystalline cellulose, microcrystalline cellulose, α-cellulose, methylcellulose, hydroxypropyl cellulose, carboxymethyl cellulose, ethyl cellulose, hydroxypropyl methyl cellulose, and cellulose acetate. In some cases, a composition or formulation can comprise a carrier in an amount of about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 97.5% 98%, 98.5%, 99%, 99.5%, or 99.9% (wt/wt) of the total composition. In some cases, a composition or formulation can comprise a carrier in an amount of about 60% to about 99.9% (wt/wt) of the total composition. In some cases, a composition or formulation can comprise one carrier. In some cases, a composition or formulation can comprise more than one carrier, for example a composition can comprise 2, 3, 4, 5, or more carriers. In some cases, a carrier can comprise a non-aqueous carrier. In some cases, a non-aqueous carrier can comprise a solvent, an excipient, a vehicle, a permeation enhancer (also known a penetration enhancer), or a mixture thereof.

In some cases, a composition herein can be an aqueous formulation or a non-aqueous formulation. In some cases, the benefits of non-aqueous solution formulations can be 1) there may not be a need to include a preservative, and 2) there can be potentially higher drug loading in the formulations due to potentially higher solubility of a PDE inhibitor such as theophylline in various non-aqueous excipients/vehicles, for example, the solubility of theophylline in water is about 7-8 mg/mL, while in ethanol, it is 14-15 mg/mL).

In some embodiments, excipients, vehicles (e.g., a carrier), or both for non-aqueous formulations (in place of water) can comprise alkylene glycols, e.g., propylene glycol, butylene glycol or any combination thereof. In some cases, excipients, vehicles (e.g., a carrier), or both for non-aqueous formulations can comprise di-alkylene glycols (e.g., dipropylene glycol). In some cases, excipients, vehicles (e.g., a carrier), or both for non-aqueous formulations can comprise Polyethylene glycols (PEG) (e.g., PEG400, PEG600, or any combination thereof). In some cases, excipients, vehicles (e.g., a carrier), or both for non-aqueous formulations can comprise unsaturated fatty alcohols, such as palmitoleyl alcohol, oleyl alcohol, erucyl alcohol, or any combination thereof. In some cases, excipients, vehicles (e.g., a carrier), or both for non-aqueous formulations can comprise DMSO, Transcutol P (Diethylene glycol monoethyl ether), ethanol, acetone, or any combination thereof.

In some embodiments, a composition or formulation can comprise a diluent. In some cases, a diluent can comprise an excipient. In some cases, a diluent can comprise a water, a saline, a buffer, a sugar, a binder, a disintegrant or any combination thereof.

In some embodiments, a composition or formulation can comprise a preservative. In some cases, a preservative can comprise an alcohol. In some cases, a preservative can comprise phenylethyl alcohol. In some cases, a preservative can comprise methylparaben, propylparaben, benzalkonium chloride, phenylcarbinol, potassium sorbate, or a combination thereof. In some cases, a composition disclosed herein can be preservative free. In some cases, a composition or formulation can comprise a preservative in an amount of about: 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, or 5% (wt/wt) of the total composition. In some cases, a composition or formulation can comprise a preservative in an amount of about 0.01% to about 10% (wt/wt) of the total composition. In some cases, a composition or formulation can comprise one preservative. In some cases, a composition or formulation can comprise more than one preservative, for example a composition can comprise 2, 3, 4, 5, or more preservatives.

In some embodiments, a composition or formulation can comprise a buffering agent. In some cases, a buffering agent can comprise potassium phosphate, sodium acetate, sodium citrate, sodium phosphate, trisodium citrate, or a combination thereof. In some cases, a composition or formulation can comprise a pH adjusting agent such as acetic acid, citric acid, hydrochloric acid, sodium hydroxide, sulfuric acid, or a combination thereof.

In some embodiments, the pH of the composition or formulation can be from 7.1 to 8.5. In some embodiments, the pH can be from 7.1 to 7.4. In some embodiments, the pH of the composition or formulation can be 7.1, 7.2, 7.3, 7.4, 7.5, 7.6, 7.7, 7.8, 7.9, 8, 8.1, 8.2, 8.3, 8.4, or 8.5. In some embodiments, the pH can be at least 7.1, 7.2, 7.3, 7.4, 7.5, 7.6, 7.7, 7.8, 7.9, 8, 8.1, 8.2, 8.3, 8.4, or 8.5. In some embodiments, the pH of the composition or formulation can be from 6 to 7. In some embodiments, the pH of the composition or formulation can be 6.0, 6.1, 6.2, 6.3, 6.4, 6.5, 6.6, 6.7, 6.8, 6.9, or 7. In some embodiments, the pH of the dosage unit can be from 7.1 to 8.5. In some embodiments, the pH of the dosage unit can be from 7.1 to 7.4. In some embodiments, the pH of the dosage unit can be 7.1, 7.2, 7.3, 7.4, 7.5, 7.6, 7.7, 7.8, 7.9, 8, 8.1, 8.2, 8.3, 8.4, or 8.5. In some embodiments, the pH of the dosage unit can be at least 7.1, 7.2, 7.3, 7.4, 7.5, 7.6, 7.7, 7.8, 7.9, 8, 8.1, 8.2, 8.3, 8.4, or 8.5.

In some embodiments, a composition or formulation can comprise glycerol. In some cases, a composition or formulation can comprise a flavoring agent, such as menthol, an artificial flavor (e.g., strawberry, a fruit flavor, a mint flavor), a natural flavor, saccharin sodium, sorbitol or a combination thereof. In some cases, a composition or formulation can comprise a fluorescent agent (e.g., a fluorophore). A fluorescent agent can be used in a nasal cast scan. Some examples of fluorescent agents include alexa fluor 350, alexa fluor 405, alexa fluor 488, alexa fluor 532, alexa fluor 546, alexa fluor 555, alexa fluor 561, alexa fluor 568, alexa fluor 594, alexa fluor 647, alexa fluor 660, alexa fluor 680, alexa fluor 700, alexa fluor 750, bodipy fl, coumarin, cy3, cy5, fluorescein (FITC) oregon green, pacific blue, pacific green, pacific orange, pe-cyanine 7, percp-cyanine 5.5, tetramethylrhodamine (TRITC), texas red, efluor 450, efluor 506, efluor 660, pe-efluor 610, percp-efluor 710, apc-efluor 780, super bright 436, super bright 600, super bright 645, super bright 702, super bright 780, Cyan Fluorescent Protein (CFP), Green Fluorescent Protein (GFP), Red Fluorescent Protein (RFP) or any combination thereof.

In some embodiments, a composition or formulation can comprise a humectant. In some cases, a humectant can comprise a glycerin, a propylene glycol, a hexylene glycol, a butylene glycol, a glyceryl triacetate, a vinyl alcohol, a neoagarobiose, a glycerol, a sorbitol, a xylitol, a maltitol, a polydextrose, a quillaia, a lactic acid, a urea, or an aloe vera.

In some embodiments, the compositions disclosed herein are stable in a freezer (e.g., −80° C. to about −20° C.), a refrigerator (e.g. 4° C.), or at room temperature. In some cases, a composition described herein can be stored in a container. For example, a container containing the pharmaceutical composition of a spray device. In some cases, a container is glass, plastic, metal, or any solid material. In some cases, a container is comprised in a kit. In some instances, when a sealed container containing a composition described herein is placed at about: 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., or 38° C., and a room atmosphere having about 50% or about 40%-60% relative humidity the composition can retain greater than about: 40%, 50%, 60%, 70%, 80%, 90% or 99% of the active ingredient or salt thereof after 3 months, 6 months, or 12 months, as measured by a high-performance liquid chromatography (HPLC).

In some embodiments, a composition or formulation described herein can comprise a penetration enhancer. In some cases, a penetration enhancer can be a substance that is capable of promoting penetration of a drug into the skin, mucous membrane, nerve sheath or through another barrier (e.g., a mucosal tissue). In some cases, a penetration enhancer can be at least partially: inert, non-toxic, non-irritating, non-allergenic, compatible with a drug and excipients, odorless, tasteless, colorless or any combination thereof. In some cases, a penetration enhancer can be a fatty acid, an alcohol, a surfactant, a solvent, a hydrogen bond acceptor or any combination thereof. In some instances, a penetration enhancer can comprise an azonem (1-dodecylazacycloheptan-2-one), dimethylsulfoxide, dimethylacetamide, dimethylformamide, ethanol, propylene glycol, N-methyl pyrrolidone, oleic acid, a lauryl alcohol, a ketone terpene, a terpene, a sulfoxide, an alkanol, an organic acid, an alcohol, a polyol, pyrrolidone, a glycol, urea and derivatives of urea, an enzyme, a iminosulfurane, a cyclodextrin, a fatty acid ester, a surfactant, a polymer, a monoolein, a oxalidinone, or any combination thereof. In some cases, a penetration enhancer can comprise cyclodextrin, sodium hyaluronate, cremophor RH40, chitosan, cyclopentyladenosine, a dextran, or any combination thereof. In some cases, a formulation herein can comprise an enzyme modulator.

In some embodiments, a composition or formulation can comprise a penetration enhancer in an amount from about 0.1% to about 20% weight to weight (wt/wt) of the total composition. In some cases, a composition or formulation can comprise a penetration enhancer in an amount from about: 1% to about 10%, 2% to about 8%, 5% to about 15%, 4% to about 12% or 10% to about 20% (wt/wt) of the total composition. In some cases, a composition or formulation can comprise a penetration enhancer in an amount of about: 0.1%, 0.2%. 0.3%. 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% (wt/wt) of the total composition. In some cases, a composition or formulation can comprise one penetration enhancer. In some cases, a composition or formulation can comprise more than one penetration enhancer, for example a composition or formulation can comprise 2, 3, 4, 5, or more different penetration enhancers.

In some cases, an enzyme modulator can comprise a p-glycoprotein inhibitor, a CYP450 inhibitor, an acetazolamide inhibitor, or any combination thereof. In some cases, a formulation herein can comprise a vasoconstrictor. In some cases, a vasoconstrictor can comprise a phenylephrine or a salt thereof. In some cases, a formulation can comprise a mucoadhesive. In some cases, a mucoadhesive can comprise a chitosan, a derivative of chitosan, carboxymethylcellulose, a polacrylic acid, or any combination thereof. In some cases, a formulation herein can comprise a ciliostatic. In some cases, a ciliosatic can comprise chlorbutol, a hydroxybenzoate, a chlorocresol edetate, a phenylmercuric acetate, a thiomersal, a salt of any of these, or any combination thereof.

### Nasal Spray Device

Disclosed herein are devices for administering active ingredients for treating chemosensory dysfunction in a subject such as a human. In some embodiments, the devices can be used to administer a nasal spray to a subject in need thereof. In some embodiments, the device upon actuation administers a plume of liquid droplets. In some cases, a plume of liquid droplets can comprise a dosage unit. For example, a pharmaceutical dosage unit can be administered by a device described herein. In some instances, the droplets can be referred to as particles. Upon actuation the plume can deliver the droplets to the nasal cavity. In some embodiments, the spray can be configured to deposit the droplets in the olfactory region of the nasal cavity. The spray device disclosed herein can emit particles in the size of about 10 μm to about 20 μm.

In some embodiments, the nasal spray device upon actuation delivers a plume as a unit dose. In some case, the nasal spray device comprises about 60 to about 120 unit doses. In some case, the nasal spray device comprises about: 1 to about 200 unit doses, 10 to about 250 unit doses, 5 to about 50 unit doses, 50 to about 100 unit doses, 20 to about 220 unit doses, or about 100 to about 240 unit doses. In some cases, each unit dose can comprise about 20 μg to about 4000 μg of the PDE inhibitor or the salt thereof. In some embodiments, a liquid pharmaceutical composition can be administered in the form of a plume from actuation of a device.

In some embodiments, the nasal spray device can comprise a spray nozzle. In some instances, a spray nozzle can be configured to generate a plume. In some instances, a nasal spray device can comprise one spray nozzle. In some instances, a nasal spray device can comprise multiple spray nozzles to generate a plume, for example a spray device can comprise 2, 3, 4, 5, 6, 7, 8, 9, 10, 20, 30, 40, 50 or more nozzles to generate a plume. In some cases, the spray nozzle can modify the droplet parameters such that a droplet size range can be acquired. In some instances, the droplet size range can increase or decrease the amount of drug deposited in the nasal cavity. In some instances, the droplet size range can increase or decrease the amount of coverage of a drug in a nasal cavity. In some instances, the droplet size range can increase or decrease the amount of a drug in a region of the nasal cavity. For example, a droplet size range can be acquired that deposits most of the drug in the olfactory region of the nasal cavity. In some embodiments, a nasal spray device can comprise one or more dosage units. In some cases, a nasal spray device can comprise a reservoir with a plurality of dosage units. In some embodiments, the spray device can emit a plume with a D₁₀, D₅₀, D₉₀ and span measurements. In some cases, a measurement of a droplet or particle size (e.g., D₁₀, D₅₀, or D₉₀ values) can be determined by a laser diffraction system. For example, a Malvern Spraytec system.

In some embodiments, a spray device can comprise one or more nozzle pores. In some cases, a spray device can comprise a nozzle pore size of about: 1 μm, 2 μm, 3 μm, 4 μm, 5 μm, 6 μm, 7 μm, 8 μm, 9 μm, or 10 μm. In some cases, a spray device can comprise a nozzle pore size of 3 μm. In some cases, a spray device can comprise a nozzle pore size of 4 μm. In some cases, a spray device can comprise a nozzle pore size of 5 μm. In some cases, a spray device can comprise about: 10 to about 200 nozzle pores, 10 to about 100 nozzle pores, 10 to about 60 nozzle pores, 20 to about 80 nozzle pores, 30 to about 100 nozzle pores, 40 to about 70 nozzle pores, 45 to about 65 nozzle pores, or 48 pores to about 60 nozzle pores. In some cases, a spray device can comprise about: 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nozzle pores. In some cases, a spray device can comprise 60 nozzle pores. In some cases, a spray device can comprise 48 pores. In some cases, a spray device can comprise a cone angle for administration of the spray at a given angle. In some cases, a cone angle of a spray device can be about: 10° to about 80°, 10° to about 60°, 10° to about 40°, 10° to about 30°, 15° to about 25°, or 20° to about 40°. In some cases, a cone angle of a spray device can be about: 1°, 2°, 3°, 4°, 5°, 6°, 7°, 8°, 9°, 10°, 11°, 12°, 13°, 14°, 15°, 16°, 1°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, or 80°. In some cases, a cone angle of a spray device can be about 20°.

In one example, a spray device can have 60 nozzle pores at a size of 4 μm per pore with a 20° cone angle. In another example, a spray device can have 48 nozzle pores at a size of 5 μm per pore with a 20° cone angle. In another example, a spray device can have 60 nozzle pores at a size of 5 μm per pore with a 20° cone angle. In another example, a spray device can have 48 nozzle pores at a size of 4 μm per pore with a 20° cone angle.

In some embodiments, a volume of the dosage unit can be from about 25 μL to about 2000 μL. In some embodiments, a spray device herein can comprise a single dosage unit or can comprise multiple dosage units. For example, a spray device described herein can be a multi-dose spray unit. In some cases, an actuation of a spray device can dispense a volume of a dosage unit. In some cases, a volume of the dosage unit can be from about 50 μL to about 400 μL. In some cases, a volume of the dosage unit can be from about: 20 μL to about 80 μL, 40 μL to about 300 μL, 60 μL to about 350 μL, 80 μL to about 450 μL, 100 μL to about 600 μL, 250 μL to about 600 μL, 500 μL to about 1 mL, 750 μL to about 1.5 mL, or about 1 mL to about 2 mL. In cases, a volume of the dosage unit can be about: 20 μL, 21 μL, 22 μL, 23 μL, 24 μL, 25 μL, 26 μL, 27 μL, 28 μL, 29 μL, 30 μL, 31 μL, 32 μL, 33 μL, 34 μL, 35 μL, 36 μL, 37 μL, 38 μL, 39 μL, 40 μL, 41 μL, 42 μL, 43 μL, 44 μL, 45 μL, 46 μL, 47 μL, 48 μL, 49 μL, 50 μL, 51 μL, 52 μL, 53 μL, 54 μL, 55 μL, 56 μL, 57 μL, 58 μL, 59 μL, 60 μL, 61 μL, 62 μL, 63 μL, 64 μL, 65 μL, 66 μL, 67 μL, 68 μL, 69 μL, 70 μL, 71 μL, 72 μL, 73 μL, 74 μL, 75 μL, 76 μL, 77 μL, 78 μL, 79 μL, 80 μL, 81 μL, 82 μL, 83 μL, 84 μL, 85 μL, 86 μL, 87 μL, 88 μL, 89 μL, 90 μL, 91 μL, 92 μL, 93 μL, 94 μL, 95 μL, 96 μL, 97 μL, 98 μL, 99 μL, or 100 μL. In some cases, a volume of the dosage unit can be about: 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, 250 μL, 260 μL, 270 μL, 280 μL, 290 μL, 300 μL, 310 μL, 320 μL, 330 μL, 340 μL, 350 μL, 360 μL, 370 μL, 380 μL, 390 μL, 400 μL, 410 μL, 420 μL, 430 μL, 440 μL, 450 μL, 460 μL, 470 μL, 480 μL, 490 μL, 500 μL, 510 μL, 520 μL, 530 μL, 540 μL, 550 μL, 560 μL, 570 μL, 580 μL, 590 μL, or 600 μL. In some cases, a volume of the dosage unit can be about: 600 μL, 700 μL, 800 μL, 900 μL, 1000 μL, 1100 μL, 1200 μL, 1300 μL, 1400 μL, 1500 μL, 1600 μL, 1700 μL, 1800 μL, 1900 μL, or 2000 μL.

In some embodiments, a dosage unit can be in the form of a plume having a droplet size distribution characterized by one or more of the following: (a) less than about 3% of the droplets in the plume having a size of less than about 10 μm, (b) a D₁₀ of less than about 15 μm, wherein about 10% of the droplets in the plume have a size less than the D₁₀, (c) a D₅₀ of from about 15 to about 24 μm, wherein about 50% of the droplets in the plume have a size less than the D₅₀, (d) a D₉₀ of from about 30 μm to about 50 μm, wherein about 90% of the droplets in the plume have a size less than the D₉₀, and (e) a span of from about 1 to about 4, wherein the span is calculated according to: (D₉₀−D₁₀)/D₅₀.

In some embodiments, a dosage unit can be in the form of a plume having a droplet size distribution characterized by one or more of the following: (a) less than about 1.5% of the droplets in the plume having a size of less than about 10 μm, (b) a D₁₀ of less than about 20 μm, wherein about 10% of the droplets in the plume have a size less than the D₁₀, (c) a D₅₀ of from about 23 μm to about 30 μm, wherein about 50% of the droplets in the plume have a size less than the D₅₀, (d) a D₉₀ of from about 38 μm to about 48 μm, wherein about 90% of the droplets in the plume have a size less than the D₉₀, and (e) a span of from about 0.9 to about 1.4, wherein the span is calculated according to: (D₉₀−D₁₀)/D₅₀.

In some embodiments, a plume can be characterized by less than about 4% of the droplets in the plume having a size of less than about 10 μm. In some embodiments, the plume can be characterized by less than about 3% of the droplets in the plume having a size of less than about 10 μm. In some embodiments, the plume can be characterized by less than about 2% of the droplets in the plume having a size of less than about 10 μm. In some embodiments, the plume can be characterized by less than about 1.5% of the droplets in the plume having a size of less than about 10 μm. In some embodiments, the plume can be characterized by less than about 1% of the droplets in the plume having a size of less than about 10 μm. In some embodiments, the plume droplets can be measured from the total amount of droplets in a plume. In some embodiments, the plume droplets can be measured from the total volume of droplets in a plume.

In some embodiments, a plume can be characterized by less than about 3% of the droplets in the plume having a size of less than about 5 μm. In some embodiments, the plume can be characterized by less than about 2% of the droplets in the plume having a size of less than about 5 μm. In some embodiments, the plume can be characterized by less than about 1% of the droplets in the plume having a size of less than about 5 μm. In some embodiments, the plume can be characterized by less than about 0.5% of the droplets in the plume having a size of less than about 5 μm. In some embodiments, the plume can be characterized by less than about 0.25% of the droplets in the plume having a size of less than about 5 μm. In some embodiments, the plume can be characterized by substantially all or all of the droplets in the plume being larger than about 5 μm. In some embodiments, the plume droplets can be measured from the total amount of droplets in a plume. In some embodiments, the plume droplets can be measured from the total volume of droplets in a plume.

In some embodiments, a D₁₀ comprises about 10% of the droplets in a plume having a size less than the D₁₀. In some embodiments, the plume can be characterized by a D₁₀ that can be less than about 20 μm. In some embodiments, the plume can be characterized by a D₁₀ that can be less than about 15 μm. In some cases, the plume can be characterized by a D₁₀ that can be from about 10 μm to about 20 μm. In some cases, the plume can be characterized by a D₁₀ that can be from about 8 μm to about 15 μm. In some cases, the plume can be characterized by a D₁₀ that can be from about 12 μm to about 19 μm. In some cases, the plume can be characterized by a D₁₀ that can be about: 8 μm, 9 μm, 10 μm, 11 μm, 12 μm, 13 μm, 14 μm, 15 μm, 16 μm, 17 μm, 18 μm, 19 μm, or 20 μm. In some cases, the plume can be characterized by a D₁₀ that can be greater than about: 8 μm, 9 μm, 10 μm, 11 μm, 12 μm, 13 μm, 14 μm, 15 μm, 16 μm, 17 μm, 18 μm, 19 μm, or 20 μm.

In some embodiments, the D₅₀ comprises about 50% of the droplets in a plume having a size less than the D₅₀. In some embodiments, the plume can be characterized by the D₅₀ that can be from about 15 μm to about 24 μm. In some embodiments, the plume can be characterized by the D₅₀ that can be from about 10 μm to about 20 μm. In some embodiments, the plume can be characterized by the D₅₀ that can be from about 20 μm to about 30 μm. In some embodiments, the plume can be characterized by the D₅₀ that can be from about 23 μm to about 30 μm. In some embodiments, the plume can be characterized by the D₅₀ that can be about: 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, or 33 μm.

In some embodiments, the D₉₀ comprises about 90% of the droplets in a plume having a size less than the D₉₀. In some embodiments, the plume can be characterized by the D₉₀ that can be less than about 65 μm. In some embodiments, the plume can be characterized by the D₉₀ that can be less than about 60 μm. In some embodiments, the plume can be characterized by the D₉₀ that can be less than about 50 μm. In some embodiments, the plume can be characterized by the D₉₀ that can be less than about 40 μm. In some embodiments, the plume can be characterized by the D₉₀ that can be less than about 35 μm. In some embodiments, the plume can be characterized by the D₉₀ that can be less than about: 60 μm, 59 μm, 58 μm, 57 μm, 56 μm, 55 μm, 54 μm, 53 μm, 52 μm, 51 μm, 50 μm, 49 μm, 48 μm, 47 μm, 46 μm, 47 μm, 46 μm, 45 μm, 44 μm, 43 μm, 42 μm, 41 μm, 40 μm, 39 μm, 38 μm, 37 μm, 36 μm, 35 μm, 34 μm, 33 μm, 32 μm, 31 μm, 30 μm, 29 μm, or 28 μm. In some embodiments, the plume can be characterized by a D₉₀ of less than about 35 μm, or a D₉₀ of less than about 42 μm. In some embodiments, the plume can be characterized by a D₉₀ of less than about 49 μm. In some embodiments, the plume can be characterized by the D₉₀ that can be from about 50 μm to about 60 μm. In some embodiments, the plume can be characterized by the D₉₀ that can be from about 45 μm to about 55 μm. In some embodiments, the plume can be characterized by the D₉₀ that can be from about 30 μm to about 50 μm. In some embodiments, the plume can be characterized by the D₉₀ that can be from about 40 μm to about 50 μm. In some embodiments, the plume can be characterized by the D₉₀ that can be from about 38 μm to about 49 μm. In some embodiments, the plume can be characterized by the D₉₀ that can be from about 35 μm to about 45 μm. In some embodiments, the plume can be characterized by the D₉₀ that can be from about 30 μm to about 40 μm. In some embodiments, the plume can be characterized by the D₉₀ that can be from about 25 μm to about 35 μm. In some embodiments, the plume can be characterized by the D₉₀ that can be about: 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm or 65 μm.

In some embodiments, the plume can be characterized by the span that can be from about 1 to about 5. In some embodiments, the plume can be characterized by the span that can be from about 1 to about 4. In some embodiments, the plume can be characterized by the span that can be from about 1 to about 3. In some embodiments, the plume can be characterized by the span that can be from about 1 to about 2. In some embodiments, the plume can be characterized by the span that can be from about 0.5 to about 1. In some embodiments, the plume can be characterized by the span that can be from about 0.5 to about 1.5. In some embodiments, the plume can be characterized by the span that can be about: 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.7, 2.9, 3, 3.25, 3.5, 3.75, 4, 4.5, 5, 5.5, or 6.

In some embodiments, the plume can be characterized by having an ovality of, for example, from about 0.5 to about 2. In some embodiments, the plume can be characterized by having an ovality from about 0.5 to about 1. In some embodiments, the plume can be characterized by having an ovality from about 0.8 to about 1.5. In some embodiments, the plume can be characterized by having an ovality from about 1 to about 1.5. In some embodiments, the plume can be characterized by having an ovality from about 1.1 to about 1.8. In some embodiments, the plume can be characterized by having an ovality of about: 0.5, 0.6, 0.7, 0.8, 0.9, 1, 1.1, 1.2, 1.3, 1.4, 1.5, 1.6, 1.7, 1.8, 1.9, or 2.0.

In some embodiments, the plume can be characterized by having a geometry (e.g., plume angle) from about 10° to about 90°. In some embodiments, the plume can be characterized by having a geometry from 45° to 75°. In some embodiments, the plume can be characterized by having a geometry from 20° to 45°. In some embodiments, the plume can be characterized by having a geometry from 15° to 40°. In some embodiments, the plume can be characterized by having a geometry from 25° to 40°. In some embodiments, the plume can be characterized by having a geometry of about: 10°, 15°, 20°, 25°, 30°, 35°, 40°, 45°, 50°, 55°, 60°, 65°, 70°, 75°, 80°, 85°, or 90°.

In some embodiments, the plume can be characterized by having a plume width from about 10 mm to about 90 mm. In some embodiments, the plume can be characterized by having a plume width from 45 mm to 75 mm. In some embodiments, the plume can be characterized by having a plume width from 20 mm to 45 mm. In some embodiments, the plume can be characterized by having a plume width from 15 mm to 40 mm. In some embodiments, the plume can be characterized by having a plume width from 25 mm to 40 mm. In some embodiments, the plume can be characterized by having a plume width of about: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, or 90 mm.

In some cases, the plume can be characterized by having a maximum diameter of the spray (Dmax) of about 10 mm to about 60 mm. In some cases, the plume can be characterized by having a Dmax of about 10 mm to about 50 mm. In some cases, the plume can be characterized by having a Dmax of about 10 mm to about 40 mm. In some cases, the plume can be characterized by having a Dmax of about 10 mm to about 30 mm. In some cases, the plume can be characterized by having a Dmax of about 10 mm to about 20 mm. In some cases, the plume can be characterized by having a Dmax of about 15 mm to about 20 mm. In some cases, the plume can be characterized by having a Dmax of about 16 mm to about 20 mm. In some cases, the plume can be characterized by having a Dmax of about 20 mm to about 50 mm. In some cases, the plume can be characterized by having a Dmax of about 30 mm to about 45 mm. In some cases, the plume can be characterized by having a Dmax of about 30 mm to about 50 mm. In some embodiments, the plume can be characterized by having a Dmax of about: 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, or 60 mm.

In some cases, the plume can be characterized by having a minimum diameter of the spray (Dmin) of about 10 mm to about 40 mm. In some cases, the plume can be characterized by having a Dmin of about 10 mm to about 30 mm. In some cases, the plume can be characterized by having a Dmin of about 10 mm to about 20 mm. In some cases, the plume can be characterized by having a Dmin of about 10 mm to about 15 mm. In some cases, the plume can be characterized by having a Dmin of about 20 mm to about 35 mm. In some cases, the plume can be characterized by having a Dmin of about 25 mm to about 35 mm. In some cases, the plume can be characterized by having a Dmin of about 21 mm to about 32 mm. In some embodiments, the plume can be characterized by having a Dmin of about: 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, or 40 mm.

In some embodiments, the plume can be characterized by having a plume area from about 100 mm² to about 1500 mm². In some embodiments, the plume can be characterized by having a plume area from 500 mm² to 1200 mm². In some embodiments, the plume can be characterized by having a plume area from 600 mm² to 1300 mm². In some embodiments, the plume can be characterized by having a plume area from 150 mm² to 200 mm². In some embodiments, the plume can be characterized by having a plume area from 100 mm² to 500 mm². In some embodiments, the plume can be characterized by having a plume area of about: 100 mm², 200 mm², 300 mm², 400 mm², 500 mm², 600 mm², 700 mm², 800 mm², 900 mm², 1000 mm², 1100 mm², 1200 mm², 1300 mm², 1400 mm², or 1500 mm².

In some embodiments, a spray device can have an actuation time, which can be from the start of the spray to the end of the spray. In some cases, the spray device can have an actuation time of 0.5 seconds to about 5 seconds. In some cases, a spray device can have an actuation time of about 3 seconds. In some cases, a spray device can have an actuation time of about: 0.5 seconds, 1 second, 1.5 seconds, 2 seconds, 2.5 seconds, 3 seconds, 3.5 seconds, 4 seconds, 4.5 seconds or about 5 seconds. In some instances, an increase in an actuation time can increase the amount of the spray deposited on an olfactory region.

In some embodiments, a spray device can have a length of actuation. In some cases, the length of actuation can be about: 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, or 5.5 mm. In some cases, the length of actuation can be about 4.6 mm, 4.8 mm or 4.9 mm.

In some embodiments, a spray device can have an actuation stroke acceleration of about: 100 millimeters/second² (mm/s²), 200 mm/s², 300 mm/s², 400 mm/s², 500 mm/s², 600 mm/s², 700 mm/s², 800 mm/s², 900 mm/s², or 1000 mm/s². In some cases, the actuation stroke acceleration of a spray device can be about 500 mm/s².

In some embodiments, a spray device can have an actuation stroke velocity of about: 0.5 millimeters/second (mm/s), 1 mm/s, 2 mm/s, 3 mm/s, 4 mm/s, 5 mm/s, 6 mm/s, 7 mm/s, 8 mm/s, 9 mm/s, or 10 mm/s. In some cases, a spray device can have an actuation stroke velocity of about: 1 mm/s, 2 mm/s, 3 mm/s, or 4 mm/s.

In some cases, a spray device can have an average hold time of about 300 milliseconds (ms), 400 ms, 500 ms, 600 ms, 700 ms, 800 ms, 900 ms, or 1000 ms. In some cases, a spray device can have an average hold time of about 684 ms.

In some cases, a spray device can deliver a shot weight per actuation. In some cases, a delivered shot weight can be about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg per actuation. In some cases, a delivered shot weight can be about: 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per actuation. In some cases, a delivered shot weight can be about 45 mg or within about: 10%, 15%, 20%, or 25% of 45 mg.

In some cases, a spray device can deliver a metered a shot weight per actuation. In some cases, a metered shot weight can be about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg per actuation. In some cases, a metered shot weight can be about: 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per actuation. In some cases, a metered shot weight can be about 45 mg or within about: 10%, 15%, 20%, or 25% of 45 mg.

In some cases, a spray device can be tested by an automated system. In some cases, a spray device can be primed before testing. For example, a spray device may be actuated several times to ensure the spray device contains a full dose prior to testing. In some case, the characteristics of a spray (e.g., a plume) can be determined at a distance. In some cases, the characteristics of a plume can be determined at about: 10 mm, 20 mm, 30 mm, 40 mm 50 mm 60 mm, 70 mm, 80 mm, 90 mm, or 100 mm. In some cases, the characteristics of a plume can be determined at about 30 mm or about 60 mm.

### Methods of Treatment

Disclosed herein are methods for treating chemosensory dysfunction in a human. In some embodiments, the method of treatment can comprise administering a nasal spray to a subject in need thereof. In some cases, disclosed herein are methods for restoring proper chemosensory function in a subject who has chemosensory dysfunction. In some cases, the chemosensory dysfunction can be at least in part produced from, or occurring during or after, a viral infection (e.g., a coronavirus infection, an influenza infection). In some cases, chemosensory dysfunction can be at least in part produced from an allergy, an inflammation, a traumatic accident, a neurodegenerative disorder, or an idiopathic cause. In some cases, the chemosensory dysfunction can be at least in part produced from, or occurring during or after, damage to the nervous system (e.g., the sensory nervous system). In some cases, the chemosensory dysfunction can be at least in part produced from, or occurring during or after, a coronavirus infection or a mutated form thereof. In some cases, a coronavirus can comprise SARS-CoV-2 or a mutated form thereof, which can cause the disease COVID-19. In some cases, the method can comprise administering to a subject a therapeutically effective amount of a phosphodiesterase (PDE) inhibitor to treat chemosensory dysfunction. In some cases, a PDE inhibitor can be administered in the form of an intranasal formulation, for example a spray formulated to deposit droplets comprising the PDE inhibitor into the nasal cavity. In some cases, a PDE inhibitor can be administered in a formulation in unit dose form.

In some embodiments, the nasal spray device herein can provide enhance delivery of a therapeutic by bypassing the blood brain barrier (BBB). A therapeutic administered herein can travel to the CNS by crossing the epithelium in either the olfactory region (OR) and move along the olfactory nerve to the olfactory bulb, or the lateral respiratory regions and the trigeminal nerve to the pons. In some cases, trigeminal neuronal endings may only be found within the lower regions of the epithelia, may not directly be exposed to the nasal cavity. Olfactory neuron cell bodies can be found within the epithelia and their cilia can reach directly into the nasal cavity. In some cases, a therapeutic disclosed herein can be transported to the CNS by an intracellular transport mechanism, for example internalization of the therapeutic by the neuron at the site of the epithelium, transport along the axon and exocytosis at the within the CNS. In some cases, a therapeutic disclosed herein can cross the epithelium via paracellular transport. In some instances, a therapeutic disclosed herein access the CNS by systemic circulation. In some cases, a therapeutic disclosed herein can be transported to the CNS by an extracellular transport mechanism, for example the therapeutic moving through fluid in the spaces along which the neurons run to the CNS.

In some cases, the method of treating a chemosensory dysfunction in a subject in need thereof can comprise administering a phosphodiesterase (PDE) inhibitor using a nasal spray device that delivers a dosage unit in a plume upon actuation. In some instances, the dosage unit can comprise a therapeutically effective amount of a PDE inhibitor in a pharmaceutically acceptable carrier, diluent, excipient, or any combination thereof. In some cases, the plume can have a droplet size distribution characterized by: (a) less than about 1.5% of the droplets in the plume having a size of less than about 10 μm; and (b) a D₉₀ of from about 38 μm to about 49 μm.

In some cases, the method of treating a chemosensory dysfunction in a subject in need thereof can comprise administering to the subject an effective amount of a pharmaceutical composition that comprises: a phosphodiesterase (PDE) inhibitor and a pharmaceutically acceptable carrier, excipient, diluent, or any combination thereof. In some cases, the pharmaceutical composition, upon intranasal administration to the subject by actuation of a nasal spray device that comprises the liquid pharmaceutical composition, forms a plume comprising a plurality of droplets that can be characterized by: a D₉₀ of from about 38 μm to about 49 μm, wherein about 90% of the droplets in the plume have a size less than the D₉₀.

In some cases, methods or compositions of the disclosure as described herein can be used to treat a subject inflicted with a chemosensory disorder. A chemosensory disorder can include a loss of smell (anosmia), or a reduced ability to smell (hyposmia). A chemosensory disorder can include a loss of taste (ageusia), or a reduced ability to taste (hypogeusia), for example, a reduced ability to taste a sweet, sour, bitter, or salty substance. In some instances, a chemosensory disorder involves odors or tastes or flavors to be misread or distorted. For example, a chemosensory disorder can cause a person to detect an unpleasant odor or taste from something that is normally pleasant to taste or smell. In some cases, chemosensory dysfunction can comprise at least partial: loss of the sense of taste, loss of the sense of smell or both. Chemosensory dysfunction can comprise a taste or smell disorder. In some cases, a taste or smell disorder can comprise anosmia, hyposmia, ageusia, hypogeusia, dysosmia (distortion of normal smell), phantosmia, dysgeusia (distortion of normal taste), parosmia, or a combination thereof.

In some embodiments, a composition herein can be used to treat an asthma, a diarrhea, a motion sickness, a gastrointestinal disorder, an overactive bladder, an urinary incontinence, a poisoning, a muscle spasms, a motion sickness, a chronic obstructive pulmonary disease (COPD), a hyperhidrosis, or any combination thereof. For example, an anticholinergic agent herein can be used to treat an asthma, a diarrhea, a motion sickness, a gastrointestinal disorder, an overactive bladder, an urinary incontinence, a poisoning, a muscle spasms, a motion sickness, a chronic obstructive pulmonary disease (COPD), or a hyperhidrosis. In some embodiments, a composition herein can be used to treat a schizophrenia, a psychosis, a schizoaffective disorder, a bipolar disorder, a depression, or any combination thereof. In some cases, an anticholinergic agent such as thioridazine, haloperidol, olanzapine, or a salt of any of these can be used to treat a chemosensory dysfunction. In some cases, a chemosensory dysfunction can comprise phantosmia or phantogeusia.

In some cases, administering a PDE inhibitor as described herein can be used to prevent or treat diseases or conditions associated with or caused by a viral infection, a bacterial infection, a fungal infection, a parasitic infection, or any combination thereof. In some cases, administering a PDE inhibitor as described herein can be used to prevent or treat diseases or conditions associated with the nervous system, such as the sensory nervous system, the central nervous system, or the peripheral nervous system. In some cases, a viral infection can comprise coronavirus. In some cases, a viral infection can comprise an influenza virus. Such diseases and conditions can include, for example, anosmia, taste loss, smell loss, hyposmia, ageusia, dysosmia, parosmia, phantosmia, chemosensory dysfunction, cough, fever, fever, malaise, difficult breathing, runny nose, sore throat, nasal congestion or any combination thereof. In some cases, an influenza infection can be caused by Influenza A, Influenza B (e.g., B (Victoria), B (Yamagata)). In some cases, Influenza A can comprise Influenza H1N1, H3N2, a mutation of any of these, or any combination thereof. In some cases, an influenza virus can comprise one or more mutations. In some cases, a coronavirus infection can be caused by alpha coronavirus, beta coronavirus, gamma coronavirus, delta coronavirus, 229E coronavirus, NL63 coronavirus, OC43 coronavirus, HKU1 coronavirus, MERS-CoV, SARS-CoV, SARS-CoV-2, a mutated form thereof, or any combination of these. In some cases, a coronavirus can cause COVID-19. In some cases, a virus, such as a coronavirus or an influenza virus can have a mutation. For example, a coronavirus or an influenza virus can comprise about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotide mutations as compared to a reference sequence. In some cases, a coronavirus or an influenza virus can comprise a genome with more than about: 70%, 71%, 72%, 73%, 74%, 75%, 76%, 77%, 78%, 79%, 80%, 81%, 82%, 83%, 84%, 85%, 86%, 87%, 88%, 89%⁰, 90%, 91%, 92%, 93%, 94%, 95%, 96%, 97%, 98%, or 99% sequence identity or sequence similarity as compared to a reference genome sequence. In some cases, a coronavirus or an influenza virus can comprise a genome with less than about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity or sequence similarity as compared to a reference genome sequence. In some cases, a reference sequence can be a reference sequence from the National Center for Biotechnology Information.

A chemosensory disorder can accompany, or be associated with a disease or condition, such as, obesity, diabetes, hypertension, malnutrition, or a degenerative disease of the nervous system, such as, Parkinson's disease, Alzheimer's disease, or multiple sclerosis. In some embodiments, a chemosensory disorder can be associated with an allergy such as allergic rhinitis. In some instances, an allergy can comprise a drug allergy, a food allergy, an insect allergy, a latex allergy a mold allergy, a pet allergy, a pollen allergy, or a combination thereof. In some embodiments, a chemosensory disorder can be associated with an inflammation. In some cases, inflammation can comprise sinusitis, mucosal inflammation, rhinosinusitis, nasal polyposis or any combination thereof. In some embodiments, a chemosensory disorder can be associated with a trauma. In some cases, trauma can comprise a traumatic brain injury (TBI), a head injury, a concussion, or any combination thereof.

In some cases, PDE inhibitor as described herein can be administered to a subject with a comorbidity. Such comorbidities can include, for example, hypertension, pulmonary hypertension, congestive heart failure, renal failure, myocardial infraction, stable, unstable and variant (Prinzmetal) angina, atherosclerosis, cardiac edema, heart disease, renal insufficiency, nephrotic edema, hepatic edema, stroke, asthma, bronchitis, chronic obstructive pulmonary disease (COPD), cystic fibrosis, dementia including Alzheimer's disease, immunodeficiency, premature labor, Parkinson's disease, multiple sclerosis, dysmenorrhoea, benign prostatic hyperplasis (BPH), bladder outlet obstruction, incontinence, conditions of reduced blood vessel patency, e.g., postpercutaneous transluminal coronary angioplasty (post-PTCA), peripheral vascular disease, respiratory disease, bronchitis, emphysema, lung cancer, cystic fibrosis, pneumonia, pleural effusion, allergic rhinitis, glaucoma, malignancies and diseases characterized by disorders of gut motility (e.g., irritable bowel syndrome (IBS)), a rheumatoid arthritis, a bacterial infection, a fungal infection, a parasitic infection, a viral infection, HIV, systemic lupus erythematosus, psoriasis, other autoimmune diseases, Huntington's chorea, and Amyotrophic lateral sclerosis (ALS) or any combination thereof. Treatment of comorbidities can be accomplished by administering to a patient in need thereof a therapeutically effective amount of the compound and/or composition described herein.

### Administration and Dosing

Disclosed herein are methods of treating a condition (e.g., taste and smell loss) by administering a PDE inhibitor as described herein. In some cases, administering can comprise administering a PDE inhibitor in unit dose form, for example in a nasal spray. In some embodiments, the method of treatment can comprise nasal administration by a nasal spray device. Other methods of treatment include by way of example only, oral administration, transmucosal administration, buccal administration, nasal administration, inhalation, parental administration, intravenous, subcutaneous, intramuscular, sublingual, transdermal administration, and rectal administration.

In some cases, administration can comprise administration to a nasal cavity. In some embodiments, administration in the nasal cavity can comprise administration to a nasal septum, a nasal floor, a lateral nasal wall, an inferior meatus, a middle meatus, a superior meatus, an olfactory cleft, an olfactory region, a nasal turbinate, or any combination thereof. In some embodiments, administration in the nasal cavity can comprise administration to a nasal septum, a nasal floor, a lateral nasal wall, an inferior meatus, a middle meatus, a superior meatus, an olfactory cleft, an olfactory region, and a nasal turbinate. The nasal cavity is a space that extends from the nares to the nasopharynx. The medial boundary is the nasal septum, the inferior boundary is the nasal floor. The lateral boundary includes the lateral nasal wall including the turbinates. The spaces between the turbinates and the lateral nasal wall including the inferior meatus, middle meatus, superior meatus and sphenoethmoid recess are within the nasal cavity. The superior boundary is defined by the skull base formed from the frontal bone, the cribriform plate of the ethmoid and the sphenoid bone. The olfactory nerves can be found in the superior aspect of the nasal cavity within the olfactory region/olfactory cleft below the cribriform plate. In some embodiments, a nasal cavity can comprise an upper nasal cavity, an olfactory cleft, an olfactory epithelium or any combination thereof. In some cases, the nasal cavity can comprise the squamous mucosa, the olfactory mucosa, the respiratory mucosa, or a combination thereof. Administration of a composition described herein can comprise administration to any region of the nasal cavity. In some embodiments, the composition can be administered as a spray to at least partially cover the nasal cavity. In some cases, the administering can comprise application intranasally in one nostril or both nostrils.

In some embodiments, a nasal spray plum can cover about 5% to about 99% of the surface area of a nasal cavity upon actuation. In some embodiments, a nasal spray plum can cover about 10% to about 70% of the surface area of a nasal cavity upon actuation. In some embodiments, a nasal spray plum can cover about 15% to about 50% of the surface area of a nasal cavity upon actuation. In some embodiments, a nasal spray plum can cover more than about: 5%, 10%, 15%, 20%, 25%, 30%, 35%, 40%, 45%, 50%, 55%, 60%, 6%, 70%, 75%, 80%, 85%, 90%, or 95% of the surface area of a nasal cavity upon actuation. In some embodiments, a nasal spray plum can cover about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the surface area of a nasal cavity upon actuation.

In some cases, administration can comprise administration to a sinus cavity. In some cases, administration can comprise administration the ear, the eyes, the mouth or a combination thereof. In some instances, a sinus cavity can comprise an ethmoid sinus cavity, a maxillary sinus cavity, a frontal sinus cavity, or a sphenoid sinus cavity. In some instances, administration into a sinus cavity can comprise administration to a sinus ostium.

In some embodiments representative daily intranasal, lingual, pulmonary, topical or mucosal dosages can be from about 1.0 μg to 2000 mg per day, from about 1.0 μg to 500.0 mg per day, from about 10 μg to 100.0 mg per day, from about 10 μg to about 10 mg per day, from about 10 g to 1.0 mg per day, from about 10 g to 500 g per day, from about 20 g to about 2000 μg per day, from about 100 μg to about 10,000 μg per day, or from about 1 μg to 50 μg per day of the active ingredient (e.g., a PDE inhibitor). These ranges of dosage amounts represent total dosage amounts of the active ingredient per day for a given patient. In some embodiments, a daily administered dose can be less than about: 2000 mg per day, 1000 mg per day, 500 mg per day, 100 mg per day, 10 mg per day, 9 mg per day, 8 mg per day, 7 mg per day, 6 mg per day, 5 mg per day, 4 mg per day, 3 mg per day, 2 mg per day, 1 mg per day, 500 μg per day, 300 μg per day, 200 μg per day, 100 μg per day or 50 μg per day. In some embodiments, a daily administered dose can be at least about: 50 g, 60 g, 70 g, 80 g, 90 g, 100 g, 110 g, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1100 μg, 1200 μg, 1300 μg, 1400 μg, 1500 μg, 1600 μg, 1700 μg, 1800 μg, 1900 μg or about 2000 μg.

In some embodiments, therapeutically effective dosage of an active ingredient delivered by an intranasal device herein can be about: 2 times, 3 times, 4 times, 5 times, 6 times, 7 times, 8 times, 9 times or 10 or more times lower than a dosage of the same active ingredient delivered by a standard intranasal spray device. For example, a nasal spray device herein can deliver an active ingredient such as a PDE inhibitor or salt thereof at a dose of 20 μg and be as effective at treating a disease or condition as 50 μg of the same PDE inhibitor or salt thereof delivered via a standard nasal spray device.

In some embodiments, a representative dosage can be per naris per actuation of a spray device, or per naris per multiple actuations of a spray device. In some embodiments, a representative dosage can be for a dose of the nares. In some embodiments a representative dosage can be from about 1.0 μg to 2000 mg, from about 1.0 μg to 500.0 mg, from about 10 μg to 100.0 mg, from about 10 μg to about 10 mg, from about 10 μg to 1.0 mg, from about 10 μg to 500 μg, from about 20 μg to about 2000 μg, from about 100 μg to about 10,000 μg, or from about 1 μg to 50 g of an active ingredient (e.g., a PDE inhibitor or an anticholinergic). In some embodiments, an administered dose can be less than about: 2000 mg, 1000 mg, 500 mg, 100 mg, 10 mg, 9 mg, 8 mg, 7 mg, 6 mg, 5 mg, 4 mg, 3 mg, 2 mg, 1 mg, 500 μg, 300 μg, 200 μg, 100 μg, or 50 μg of the active ingredient. In some embodiments, an administered dose can be at least about, or equal to: 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 g, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1000 μg, 1100 μg, 1200 μg, 1300 μg, 1400 μg, 1500 μg, 1600 μg, 1700 μg, 1800 μg, 1900 μg or about 2000 μg of the active ingredient.

In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day. In some embodiments, a suitable dosage level of an active ingredient on a per kilo basis can be less than about: 10.0 mg/kg of body weight per day, 1 mg/kg of body weight per day, 500 μg/kg of body weight per day, 100 μg/kg of body weight per day, 10 μg/kg of body weight per day, or 1.0 μg/kg of body weight per day of the compound. In some embodiments, a suitable dosage level of an active ingredient on a per kilo basis can be at least about: 10.0 mg/kg of body weight per day, 1 mg/kg of body weight per day, 500 μg/kg of body weight per day, 100 μg/kg of body weight per day, 10 μg/kg of body weight per day of the active ingredient, or 1.0 μg/kg of body weight per day. In some embodiments, a suitable dosage level of an active ingredient on a per kilo basis can be about: 0.5 μg/kg of body weight, 1 μg/kg of body weight, 1.5 μg/kg of body weight, 2 μg/kg of body weight, 2.5 μg/kg of body weight, 3 μg/kg of body weight, 3.5 μg/kg of body weight, 4 μg/kg of body weight, 4.5 μg/kg of body weight, 5 μg/kg of body weight, 5.5 μg/kg of body weight, 6 μg/kg of body weight, 6.5 μg/kg of body weight, 7 μg/kg of body weight, 7.5 μg/kg of body weight, 8 μg/kg of body weight, 8.5 μg/kg of body weight, 9 μg/kg of body weight, 9.5 μg/kg of body weight, 10 μg/kg of body weight, 10.5 μg/kg of body weight, 11 μg/kg of body weight, 11.5 μg/kg of body weight, 12 μg/kg of body weight, 12.5 μg/kg of body weight, 13 μg/kg of body weight, 13.5 μg/kg of body weight, 14 μg/kg of body weight, 14.5 μg/kg of body weight, 15 μg/kg of body weight, 15.5 μg/kg of body weight, 16 μg/kg of body weight, 16.5 μg/kg of body weight, 17 μg/kg of body weight, 17.5 μg/kg of body weight, 18 μg/kg of body weight, 18.5 μg/kg of body weight, 19 μg/kg of body weight, 19.5 μg/kg of body weight, or 20 μg/kg of body weight.

In some embodiments, a one or more PDE inhibitors, or their salts, can be formulated in an intranasal formulation, for example an intranasal spray formulation. In some embodiments, the intranasal formulation can comprise a non-specific PDE inhibitor. In some embodiments, a PDE inhibitor can comprise theophylline or a salt thereof, cilostazol or a salt thereof, roflumilast or a salt thereof or any combination thereof. In some embodiments, the intranasal formulation can comprise an PDE inhibitor that is selective for a PDE subtype, for example, PDE: 1, 2, 3, 4, or 5. In some embodiments, the PDE inhibitor can be dosed at a range from about 0.001 mg to about: 1 mg, 2 mg, 3 mg, 4 mg, 5 mg, 6 mg, 7 mg, 8 mg, 9 mg, or 10 mg. In some cases, a dose (e.g., a dosage unit) of a PDE inhibitor can be about: 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, or 10 mg. In some embodiments, a formulation can be in unit dose form. In some embodiments, a formulation can contain a second active ingredient, such as a corticosteroid, or an antihistamine, or a vasoconstrictor, or any combination thereof. In some embodiments, a formulation does not contain a second active ingredient. In some cases, a formulation can be a pharmaceutical composition such as an intranasal formulation. In some cases, a composition can be a pharmaceutical composition.

In some cases, the amount administered can be the same amount administered to treat a particular disease or can be an amount lower than the amount administered to treat that particular disease. The dosage may be administered once per day or several or multiple times per day. For example, a nasal spray can be administered once per day or twice per day. In another example, a PDE inhibitor can be administered 2, 3, 4, 5, 6, 7, 8, 9, 10 or more times per day. In some cases, a dose delivered by 1, 2, 3, 4, 5, or more actuations of a multi-dose nasal spray device to a naris can deliver a therapeutically effectively amount of a composition disclosed herein. In some cases, a composition can be administered once, twice or thrice in a 24-hour period. In some cases, administration of a composition disclosed herein can be performed at least 1, 2, 3, 4, 5, 6, 7, 8, 9, 10, 11, 12, 13, 14, 15, 16, 17, 18, 19, 20, or 21 times a week. In some cases, administration of a composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more times a month. The dose used to treat a subject can produce the desired therapeutic or prophylactic effects, without producing serious side effects.

Administration of a composition disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days consecutive or nonconsecutive days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days.

Administration of a composition disclosed herein can be performed for a treatment duration of at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, or for life. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years or more.

In some cases, a composition can be administered as a single dose or as divided doses. In some cases, a composition described herein can be administered at a first time point and a second time point. In some cases, a composition can be administered such that a first administration can be administered before the other with a difference in administration time of about: 10 seconds, 20 seconds, 30 seconds, 40 seconds, 50 seconds, 60 seconds, 1 minute, 2 minutes, 3 minutes, 4 minutes, 5 minutes, 10 minutes, 30 minutes, 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some embodiments, a composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some embodiments, administration of an effective amount of a PDE inhibitor by intranasal (e.g., a spray), lingual, pulmonary, topical, or mucosal administration does not produce a detectable blood level of the PDE inhibitor. In some embodiments, administration of an effective amount of a PDE inhibitor by intranasal, lingual, pulmonary, topical, or mucosal administration produces blood concentration of the PDE inhibitor that can be less than about: 5 mg/dl, 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl. In some embodiments, administration of an effective amount of a PDE inhibitor by intranasal, lingual, pulmonary, topical, or mucosal administration produces blood concentration of the PDE inhibitor that can be more than about: 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl. In some embodiments, administration of an effective amount of a PDE inhibitor by intranasal, lingual, pulmonary, topical, or mucosal administration produces blood concentration of the PDE inhibitor that can be about: 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl.

In some cases, a composition described herein can be administered with one or more additional therapeutics. For example, a composition described herein can be administered with 1, 2, 3, 4, or 5 or more additional therapeutics. In another example, a PDE inhibitor can be administered with a second therapy. In some cases, a second therapy can be administered concurrently or consecutively. In some cases, an additional therapeutic can comprise an antihistamine, diphenhydramine, chlorpheniramine, cetirizine, desloratadine, fexofenadine, levocetirizine, loratadine, azelastine, olopatadine, ketotifen, olopatadine, pheniramine, a decongestant, pseudoephedrine, oxymetazoline, tetrahydrozoline, a corticosteroid, budesonide, fluticasone furoate, fluticasone propionate, mometasone, triamcinolone, beclomethasone, ciclesonide, budesonide, fluticasone, mometasone, fluorometholone, loteprednol, prednisone, betamethasone, desonide, hydrocortisone, mometasone, triamcinolone, cromolyn, lodoxamide, nedocromil, an immunotherapy, a salt of any of these, or any combination thereof. In some cases, a second therapy can comprise remdesivir, a salt thereof, chloroquine, a salt thereof, lopinavir, a salt thereof, ritonavir, a salt thereof, molnupiravir, a salt thereof, favilavir, a salt thereof, interferon beta, a salt thereof, an antiviral, oxygen or any combination thereof. In some cases, a second therapy can comprise peramivir, a salt thereof, zanamivir, a salt thereof, oseltamivir phosphate, oseltamivir, a salt thereof, baloxavir marboxil, a salt thereof, or any combination thereof. In some cases, an additional therapeutic can comprise nitric oxide, a steroid, a non-steroidal anti-inflammatory drug (NSAID), or any combination thereof. In some cases, an additional therapeutic can comprise an anticholinergic. In some cases, an additional therapy can be comprised in a nasal spray that comprises a PDE inhibitor.

In some cases, an anticholinergic can comprise an amitriptyline, an atropine, an aclidinium, a benztropine, a chlorpheniramine, a chlorpromazine, a clomipramine, a clozapine, a cyclobenzaprine, a cyproheptadine, a darifenacin, a desipramine, a dexchlorpheniramine, a dicyclomine, a diphenhydramine, a doxepin, a hydroxyzine, a hyoscyamine, an imipramine, a meclizine, a nortriptyline, an olanzapine, an orphenadrine, an oxybutynin, a paroxetine, a perphenazine, a prochlorperazine, a promethazine, a protriptyline, a pseudoephedrine hcl/triprolidine hcl, a scopolamine, a thioridazine, a tolterodine, a trifluoperazine, a trihexyphenidyl, a trimipramine, a salt of any of these, or any combination thereof.

### Diagnosis and Measurement of Chemosensory Dysfunction

In some cases, a subject can be diagnosed (e.g., diagnosed with chemosensory dysfunction) prior to treatment with a PDE inhibitor. In some cases, a method of treatment can comprise diagnosing chemosensory dysfunction in a subject. In some cases, the diagnosing can comprise an in vitro assay. In some cases, chemosensory dysfunction can be diagnosed by detecting sonic hedgehog at or below a threshold level in a biological sample from the human. In some cases, chemosensory dysfunction can be diagnosed by cyclic AMP (cAMP), cyclic GMP (cGMP), IL-10 or a combination thereof at, below, or above a threshold level in a biological sample from the human. In some cases, a sample can be a nasal sample or a saliva sample. In some cases, chemosensory dysfunction can be diagnosed by detecting a cyclic nucleotide level at or below a threshold level in the biological sample from the human. For example, a low level of a cyclic nucleotide in a subject's biological sample can suggest the subject has chemosensory dysfunction. Measurements for detecting level of a biological material, such as cAMP can be completed via an ELISA, a Western blot or any molecular biology assay. In some embodiments, chemosensory dysfunction can be diagnosed by an olfactory assay that measures threshold, discrimination, identification, or any combination thereof. In some cases, diagnosis of chemosensory dysfunction can comprise detecting a Recognition threshold (RT) score, a Magnitude estimation (ME) score, a Detection threshold (DT) score, a Hedonic (H) score or a combination thereof and comparing to a reference population (e.g., a population without chemosensory dysfunction). A ME score refers to a measurement of the ability of a subject to determine the strength of a stimulant such as an odorant or a tastant. A RT score refers to a measurement of the ability of a subject to recognize the identity of a stimulant, such as an odorant or a tastant. A DT score refers to a measurement of the ability of a subject to recognize exposure to a stimulant, such as an odorant or a tastant, as being pleasant or unpleasant. An H score refers to a measurement of a subject's reaction to a stimulant, such as an odorant or a tastant, as being pleasant or unpleasant.

In some embodiments, the subject can be administered an olfactory assay that measures: threshold, discrimination, identification, or any combination thereof. In some instances, an olfactory assay can be administered prior to, during, or after treatment. In some cases, a threshold assay can be used to determine the lowest concentration of an odorant that can be reliably detected. In some cases, a discrimination assay can be used to assess the ability of a subject to distinguish two or more different smells. In some cases, an identification assay can be used to assess the ability of a subject to identify a specific odor. In some cases, an olfactory test can be used to determine the efficacy of a treatment such as treatment with a nasal spray device comprising a PDE inhibitor. For example, an olfactory assay can be completed prior to treatment and after treatment to determine a measurable change in a subject's chemosensory dysfunction.

In some embodiments, the subject can be administered an olfactory assay that measures: threshold, discrimination, identification or any combination thereof. In some instances, an olfactory assay can be administered prior to, during, or after treatment. In some cases, a threshold assay can be used to determine the lowest concentration of an odorant that can be reliably detected. In some cases, a discrimination assay can be used to assess the ability of a subject to distinguish two or more different smells. In some cases, an identification assay can be used to assess the ability of a subject to identify a specific odor. In some cases, an olfactory test can be used to determine the efficacy of a treatment such as treatment with an implant comprising a PDE inhibitor. For example, an olfactory assay can be completed prior to treatment and after treatment to determine a measurable change in a subject's chemosensory dysfunction.

In some embodiments, a method of treatment can comprise olfactory training (e.g., smell training). In some cases, olfactory training, which can involve repetitive stimulation of peripheral olfactory neurons, may rely on the regenerative capacity of superior olfactory pathways. In some cases, olfactory training can comprise exposure to 1, 2, 3, 4, 5, 6 or more than 6 different odors. In some cases, olfactory training can comprise exposure to the different odors 1, 2, 3, 4 or more than 4 times per day. In some cases, olfactory training can take place for about: 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, or longer than 1 year. In some cases, olfactory training can take place for about: 7 days to about 21 days, 21 days to about 120 days, 30 days to about 90 days, 1 month to about 5 months, 2 months to about 3 months, 3 months to about 8 months, 6 months to about 1 year, 8 months to about 18 months, or 1 year to about 3 years. In some cases, organic essences of lemon, rose, eucalyptus and cloves can be used as the different odors for olfactory testing.

In some embodiments, a subject can experience a change (e.g., a decrease for effective treatment) in: a detection threshold (DT) score, a recognition threshold (RT) score, or both. In some embodiments, a subject can experience a change (e.g., an increase for effective treatment) in a magnitude estimation (ME) score. In some cases, a subject can experience a change in a hedonic (H) score. In some cases, the changes in RT score, ME score, DT score, or H score can be measured with a forced-choice, three-stimuli, stepwise-staircase technique using one or more odorants after the administration of a PDE inhibitor to a subject. In some cases, the one or more odorants comprise pyridine, nitrobenzene, thiophene, amyl acetate, or a combination thereof. In some cases, the changes in RT score, ME score, DT score, or H score can be measured with a forced-choice, three-stimuli, stepwise-staircase technique using one or more tastants testing compounds after administration of a PDE inhibitor to the subject. In some cases, the one or more tastants comprise sodium chloride (NaCl), sucrose, hydrogen chloride (HCl), urea, or a combination thereof.

In some embodiments, a subject can experience a clinically detectable improvement in taste or smell function within about: 1 week to about 6 weeks, 1 week to about 4 weeks, 2 weeks to about 5 weeks, or about 3 weeks to about 4 weeks of starting treatment. In some embodiments, a subject can experience a clinically detectable improvement in taste or smell function within about: 1 month to about 6 months, 1 month to about 4 months, 2 months to about 5 months, or about 3 months to about 4 months of starting treatment.

The Hedgehog signaling pathway is known to be a key regulator of animal development, particularly during late stages of embryogenesis and metamorphosis. Mammals have three Members of the hedgehog signaling pathway, Sonic Hedgehog (SHH), Desert Hedgehog (DHH), and Indian hedgehog (IHH). The pathway is implicated in the development of some cancers. Members of the hedgehog signaling pathway can be used in diagnosing and treating loss and/or distortion of taste or smell, e.g., hyposmia, dysosmia, anosmia, phantosmia, hypogeusia, dysgeusia, phantogeusia, and/or ageusia. For example, upon treatment with a PDE inhibitor a subject with chemosensory dysfuction can have increased levels of SHH as compared to their levels before treatment.

The one or more members of the hedgehog signaling pathway can be selected from a group consisting of: Sonic Hedgehog (SHH), Desert Hedgehog (DHH), and/or Indian hedgehog (IHH). The one or more members of the hedgehog signaling pathway can be SHH, DHH, IHH, or any combination thereof. Although a mammalian (e.g., human) hedgehog can be measured, it is also contemplated that a non-mammalian hedgehog can be measured. In some cases, chemosensory dysfunction in a subject can be determined by detecting a level of: Sonic Hedgehog (SHH) that can range from about greater than 0 μg/mL to about 8,500 μg/mL; a level of Indian hedgehog (IHH) that can range from about greater than 0 μg/mL about to 1.0 μg/mL; or a level of Desert Hedgehog (DHH) that can range from about greater than 0 μg/mL to about 5.0 μg/mL, or a combination thereof.

The levels of members of the hedgehog signaling pathway in patients exhibiting loss and/or distortion of taste or smell (e.g., hyposmia, dysosmia, anosmia, phantosmia, hypogeusia, dysgeusia, phantogeusia, and/or ageusia) can be lower than normal controls. For example, in patients suffering from loss and/or distortion of taste or smell (e.g., chemosensory dysfunction), the level of SHH, in some cases, can be about: 0 pg/mL, greater than 0 pg/mL to less than less than 1 pg/mL, 1 pg/mL to 25 pg/mL, 15 pg/mL to 30 pg/mL, 20 pg/mL to 40 pg/mL; 35 pg/mL to 50 pg/mL; 45 pg/mL to 100 pg/mL; 75 pg/mL to 150 pg/mL, 125 pg/mL to 1000 pg/mL, 900 pg/mL to 2500 pg/mL, 2000 pg/mL to 5000 pg/mL, 4000 pg/mL to 7500 pg/mL, 6000 pg/mL to 10,000 pg/mL; (b) the level of IHH can be about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, less than 1.0 pg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL; (c) the level of DHH can be about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, 0.9 pg/mL to 1.1 pg/mL, 1.0 pg/mL to 1.3 pg/mL, 1.2 pg/mL to 1.5 pg/mL, 1.4 pg/mL to 2.0 pg/mL, 1.9 pg/mL to 2.5 pg/mL, 2.4 pg/mL to 3.0 pg/mL, 2.9 pg/mL to 3.5 pg/mL, 3.4 pg/mL to 3.8 pg/mL, 3.7 pg/mL to 3.9 pg/mL, 3.85 pg/mL to 5.0 pg/mL, less than 5.0 pg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL. However, there can be some inter-patient variability because the levels of the different members of the hedgehog signaling pathway vary based on the person.

In some embodiments, administration of an effective amount of a PDE inhibitor can increase the salivary and/or nasal mucus SHH, DHH, and/or IHH levels in the human by at least about: 5%, 6%, 7%, 8%, 9%, 10, 1%, 12%, 13%, 14%, 1%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of PDE inhibitor. In some cases, the increase of the salivary and/or nasal mucus SHH, DHH, and/or IHH levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor can increase or decrease a biological compound, such as a protein or a metabolite. In some embodiments, administration of an effective amount of a PDE inhibitor can increase the salivary and/or nasal mucus cAMP or cGMP levels in the human by at least about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of the PDE inhibitor. In some cases, the increase of the salivary and/or nasal mucus cAMP or cGMP levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor can decrease the salivary and/or nasal mucus IL-10 levels in the human by at least about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of the PDE inhibitor. In some cases, the decrease of the salivary and/or nasal mucus IL-10 levels are observed after about: 1 to about 10 days, 30 to about 90 days, 15 to about 45 days, or 30 days of continuous treatment with a therapeutically effective amount of a PDE inhibitor. IL-10 levels can be measured with an enzyme-linked immunoassay (ELISA), Western blot, or other protein measurement assay.

In some embodiments, administration of an effective amount of a PDE inhibitor in the form of a liquid spray can increase taste or smell acuity. In some embodiments, an increase in taste or smell acuity can be at least about: 5%, 10%, 20%, 30%, 40%, 50%, 75%, or 100% compared to the untreated state. In some embodiments, taste or smell acuity can be increased to at least about: 5%, 10%, 15%, 20%, 25%, 30%, 35%, 40%, 45%, 50%, 55%, 60%, 65%, 70%, 75%, 80%, 85%, 90%, 95% or 100% of the acuity of normal individuals. In some cases, an increase in taste or smell acuity can be measured after about: 10 to about 20 days, 15 to about 30 days, 25 days to about 50 days, 1 month to about 6 months, 4 months to 12 months, 6 months to 18 months, or 6 months to about 3 years. In some cases, an increase in taste or smell acuity can be measured after about 30 days. In some embodiments, taste or smell acuity can be measured objectively. In some embodiments, taste or smell acuity can be measured subjectively. In some cases, smell acuity can be measured by detection threshold, recognition threshold, hedonics, magnitude estimation or any method described herein.

### Kits

For use of the therapeutic compositions described herein, kits and articles of manufacture are also described. In some embodiments, such kits include a carrier, package, or container that is compartmentalized to receive one or more blister packs, bottles, tubes, capsules, and the like. In certain embodiments, the pharmaceutical compositions are presented in a pack or dispenser device which contains one or more unit dosage forms containing a compound provided herein. In other embodiments, the pack can contain metal or plastic foil, such as a blister pack. In some embodiments, the pack contains capsules, vials, or tubes. In other embodiments, the pack or dispenser device is accompanied by instructions for administration. In some embodiments, the dispenser is disposable or single use, while in other embodiments, the dispenser is reusable. In certain embodiments, the pharmaceutical formulations are preloaded into the device. In some embodiments, a kit can comprise a spray device as disclosed herein.

In other embodiments, packaging can also be accompanied with a notice as required by a governmental agency regulating the manufacture, use, or sale of pharmaceuticals. This notice states that the drug is approved by the agency for human or veterinary administration. Such notice, for example, is the labeling approved by the U.S. Food and Drug Administration for prescription drugs, or the approved product insert. Compositions containing a compound provided herein formulated in a compatible excipient, diluent and/or carrier are also prepared, placed in an appropriate container, and labeled for treatment of an indicated condition.

The articles of manufacture provided herein may also contain an administration or dispensing device. Examples of administration devices include intranasal sprays, and inhalers. Pumps and/or spray heads (e.g., a nozzle) may be provided with the sprays and intranasal devices, or the pumps and/or spray heads may be built into the devices. Alternatively, a propellant may be included with or it may be stored within the devices. In some instances, a propellant can be used with a spray device disclosed herein.

Such kits can comprise an identifying description or label for the containers. In further embodiments, the label is on a container with letters, numbers or other characters forming the label and attached, molded or etched into the container itself; a label is associated with a container when it is present within a receptacle or carrier that also holds the container, e.g., as a package insert. In some embodiments, a label is used to indicate that the contents are to be used for a specific therapeutic application. In yet other embodiments, the label also indicates directions for use of the contents, such as in the methods described herein. In some embodiments, a set of instructions may also be included, generally in the form of a package insert. The informational material may contain instructions on how to dispense the pharmaceutical composition, including description of the type of patients who may be treated, the schedule (e.g., dose and frequency), and the like.

The disclosure also relates to a set (kit) consisting of separate packs of kits that are frequently assembled for shipping or for patient convenience, such as a weekly, biweekly or monthly supply of a medicament.

### NUMBERED EMBODIMENTS

A number of compositions, methods, kits and devices are disclosed herein. Specific exemplary embodiments of these methods, kits and devices are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A method of treating a chemosensory dysfunction in a subject in need thereof comprising administering to the subject an effective amount of a pharmaceutical composition that comprises: a phosphodiesterase (PDE) inhibitor or a salt thereof and a pharmaceutically acceptable carrier, excipient, diluent, or any combination thereof wherein the pharmaceutical composition, upon intranasal administration to the subject by a nasal spray device that comprises a plume, forms a plurality of droplets characterized by: a D₅₀ of about 15 μm to about 24 μm, wherein about 50% of the droplets in the plume have a size less than the D₅₀.

Embodiment 2. The method of embodiment 1, wherein the plurality of droplets is further characterized by less than about 3% of the droplets in the plume having a size of less than about 10 μm.

Embodiment 3. The method of embodiment 1 or 2, wherein the plurality of droplets is further characterized by a D₉₀ of from about 40 μm to about 50 μm, wherein about 90% of the droplets in the plume have a size less than the D₉₀.

Embodiment 4. The method of any one of embodiments 1-3, wherein the D₅₀ is about 20 μm.

Embodiment 5. The method of any one of embodiments 1-4, wherein a droplet size is measured by a laser diffraction.

Embodiment 6. The method of any one of embodiments 1-5, wherein the PDE inhibitor comprises a theophylline or a salt thereof, a cilostazol or a salt thereof, a roflumilast or a salt thereof or any combination thereof.

Embodiment 7. The method of embodiment 6, comprising the PDE inhibitor which comprises the theophylline or a salt thereof.

Embodiment 8. The method of any one of embodiments 1-7, wherein the plume is formed by an actuation of the nasal spray device and lasts about 0.5 seconds to about 5 seconds from a start of a spray to an end of the spray.

Embodiment 9. The method of any one of embodiments 1-8, wherein the pharmaceutically acceptable carrier comprises a water.

Embodiment 10. The method of any one of embodiments 1-9, wherein the pharmaceutical composition further comprises a viscosity enhancer.

Embodiment 11. The method of embodiment 10, wherein the viscosity enhancer comprises a cellulose.

Embodiment 12. The method of any one of embodiments 1-11, wherein the pharmaceutical composition further comprises an excipient.

Embodiment 13. The method of embodiment 12, wherein the excipient comprises a glycerol.

Embodiment 14. The method of any one of embodiments 1-13, wherein the pharmaceutical composition further comprises a preservative.

Embodiment 15. The method of embodiment 8, wherein the actuation comprises an actuation amount of about 10 μl to about 200 μl, or from about 20 μl to about 80 μl, of liquid.

Embodiment 16. The method of any one of embodiments 1-15, wherein intranasal administration is once, twice, or thrice daily to each naris.

Embodiment 17. The method of any one of embodiments 1-16, wherein the plume covers about 15% to about 50%, or about 10% to about 80%, or about 5% to about 90%, or about 5% to about 100% of a surface area of a nasal cavity as measured by a nasal cast scan.

Embodiment 18. The method of embodiment 17, wherein the nasal cavity comprises a nasal septum, a nasal floor, a lateral nasal wall, an inferior meatus, a middle meatus, a superior meatus, an olfactory cleft, an olfactory region, a nasal turbinate, or any combination thereof.

Embodiment 19. The method of any one of embodiments 1-18, wherein the pharmaceutical composition is in a dosage unit comprising the PDE inhibitor and comprises a dosage of about 20 μg to about 2000 μg.

Embodiment 20. The method of any one of embodiments 1-19, wherein the chemosensory dysfunction is ageusia, hypogeusia, dysgeusia, parosmia, phantosmia, anosmia, hyposmia, dysosmia, or any combination thereof.

Embodiment 21. The method of any one of embodiments 1-19, wherein the chemosensory dysfunction is anosmia, hyposmia, dysosmia, parosmia, or a combination thereof.

Embodiment 22. The method of any one of embodiments 1-19, wherein the chemosensory dysfunction is smell loss, taste loss, or a combination thereof.

Embodiment 23. The method of any one of embodiments 1-22, wherein the chemosensory dysfunction arises after a viral infection.

Embodiment 24. The method of embodiment 23, wherein the viral infection comprises a coronavirus infection or an influenza infection.

Embodiment 25. The method of any one of embodiments 1-24, wherein the subject prior to the administering has a decreased level of a cyclic nucleotide in a nasal mucus sample from the subject in comparison to a cyclic nucleotide level of a control population with normal chemosensory function.

Embodiment 26. The method of any one of embodiments 1-25, wherein the subject prior to the administering has a decreased level of sonic hedgehog in a nasal mucus sample from the subject in comparison to a sonic hedgehog level of a control population with normal chemosensory function.

Embodiment 27. The method of any one of embodiments 1-26, further comprising administering a second therapeutic.

Embodiment 28. The method of embodiment 27, wherein the second therapeutic is administered concurrently or consecutively with the administering.

Embodiment 29. The method of any one of embodiments 1-28, further comprising diagnosing the subject with the chemosensory dysfunction.

Embodiment 30. The method of any one of embodiments 1-28, wherein the subject was previously diagnosed with the chemosensory dysfunction.

Embodiment 31. A nasal spray device comprising: a phosphodiesterase (PDE) inhibitor or a salt thereof, wherein the nasal spray device is configured to deliver a dosage unit in a plume upon actuation, wherein the dosage unit comprises a therapeutically effective amount of the PDE inhibitor or the salt thereof in a pharmaceutically acceptable carrier comprising an excipient, wherein the plume has a droplet size distribution characterized by: (a) less than about 3% of the droplets in the plume having a size of less than about 10 μm; and (b) a D₅₀ of from about 15 to about 24 μm, wherein about 50% of the droplets in the plume have a size less than the D₅₀.

Embodiment 32. A kit comprising the nasal spray device of embodiment 31 and a container.

Embodiment 33. A method of treating a chemosensory dysfunction in a subject in need thereof comprising administering a phosphodiesterase (PDE) inhibitor or a salt thereof using a nasal spray device that delivers a dosage unit in a plume upon an actuation, that comprises a therapeutically effective amount of the PDE inhibitor of a salt thereof in a pharmaceutical composition comprising a pharmaceutically acceptable carrier, diluent, excipient, or any combination thereof, wherein the plume comprises droplets and has a droplet size distribution characterized by: (a) less than about 3% of the droplets in the plume having a size of less than about 10 μm; and (b) a D₅₀ of about 15 μm to about 24 μm, wherein about 50% of the droplets in the plume have a size less than the D₅₀.

### EXAMPLES

### Example 1: Distribution of Liquid Formulations by Different Nasal Spray Devices in a Nasal Cavity Model

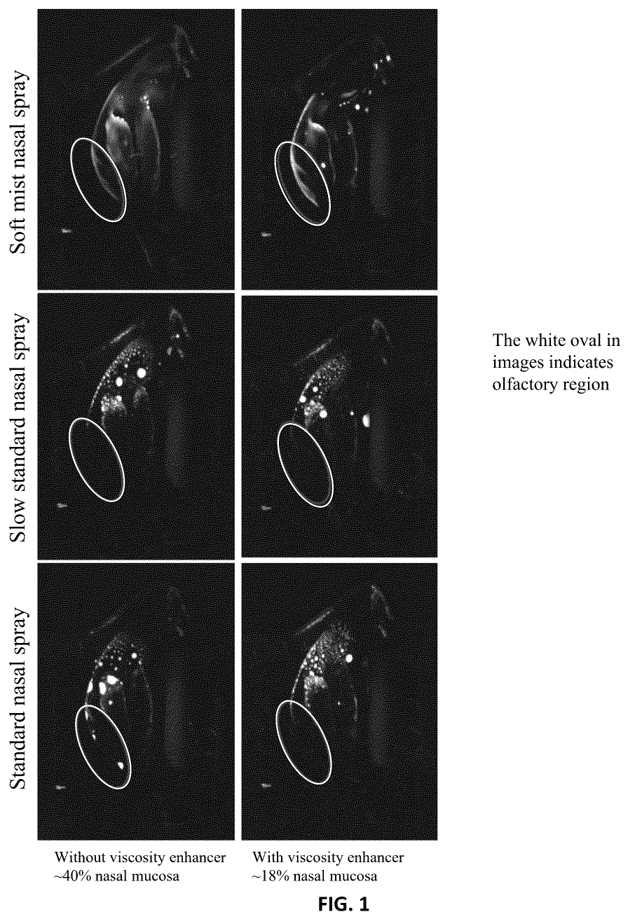

Three types of nasal spray devices were tested for their ability to deposit a liquid formulation in a nasal cavity model. 1 mg/mL calcein (for fluorescent visualization) was added to a formulation with theophylline (600 μg/mL) without a viscosity enhancer. Additionally, 1 mg/mL calcein was added to a separate formulation with theophylline (600 μg/mL) and a viscosity enhancer (carboxymethylcellulose). The formulations also comprised citric acid and sodium hydroxide as buffering agents, and phenylethyl alcohol as a preservative. Purified water was the carrier. The test was performed at 600 relative humidity. The formulations were tested in three different nasal spray devices: the soft mist nasal spray, the slow standard nasal spray, and the standard nasal spray. The images in FIG. 1 show two actuations of the spray device being tested (90 μl of liquid spray) per image. Table 1 shows the nasal coverage of different spray devices in the nasal cavity model. The soft mist nasal spray, without the viscosity enhancer had the largest surface coverage (about 40%) in the nasal cavity model. The soft mist nasal spray targeted the olfactory region and nasal turbinates. The addition of cellulose reduced the covered surface area to roughly 20% of the nasal mucosa. However, the spray was more centered to the olfactory region as shown in FIG. 1 and indicated in the oval. Additionally, the focus to the olfactory region by the soft mist nasal spray and compared to the standard nasal sprays was surprising and unexpected as this region is known to impact chemosensory function.

TABLE 1
Surface Coverage of Nasal Sprays

| Test | Nasal | | |
|---|---|---|---|
| Number | Coverage (%) | Description | Device |
| 1 | 8% | 1 mg/mL Calcein with formulation | Standard nasal spray |
| 2 | 12% | 1 mg/mL Calcein with formulation | Standard nasal spray |
| 3 | 34% | 1 mg/mL Calcein with formulation | Soft mist nasal spray |
| 4 | 43% | 1 mg/mL Calcein with formulation | Soft mist nasal spray |
| 5 | 42% | 1 mg/mL Calcein with formulation | Soft mist nasal spray |
| 6 | 10% | 1 mg/mL Calcein with formulation | Standard nasal spray |
| 7 | 13% | 1 mg/mL Calcein with formulation | Slow standard nasal spray |
| 8 | 14% | 1 mg/mL Calcein with formulation | Slow standard nasal spray |
| 9 | 11% | 1 mg/mL Calcein with formulation | Slow standard nasal spray |
| 10 | 20% | 1 mg/mL Calcein with formulation + | Soft mist nasal spray |
| | | cellulose (i.e., carboxymethylcellulose) | |
| 11 | 19% | 1 mg/mL Calcein with formulation + | Soft mist nasal spray |
| | | cellulose | |
| 12 | 18% | 1 mg/mL Calcein with formulation + | Soft mist nasal spray |
| | | cellulose | |
| 13 | 11% | 1 mg/mL Calcein with formulation + | Standard nasal spray |
| | | cellulose | |
| 14 | 12% | 1 mg/mL Calcein with formulation + | Standard nasal spray |
| | | cellulose | |
| 15 | 15% | 1 mg/mL Calcein with formulation + | Standard nasal spray |
| | | cellulose | |
| 16 | 12% | 1 mg/mL Calcein with formulation + | Standard nasal spray |
| | | cellulose | |
| 17 | 16% | 1 mg/mL Calcein with formulation + | Slow standard nasal spray |
| | | cellulose | |
| 18 | 18% | 1 mg/mL Calcein with formulation + | Slow standard nasal spray |
| | | cellulose | |
| 19 | 14% | 1 mg/mL Calcein with formulation + | Slow standard nasal spray |
| | | cellulose | |

### Example 2: Nasal Spray Characteristics of Three Nasal Spray Devices

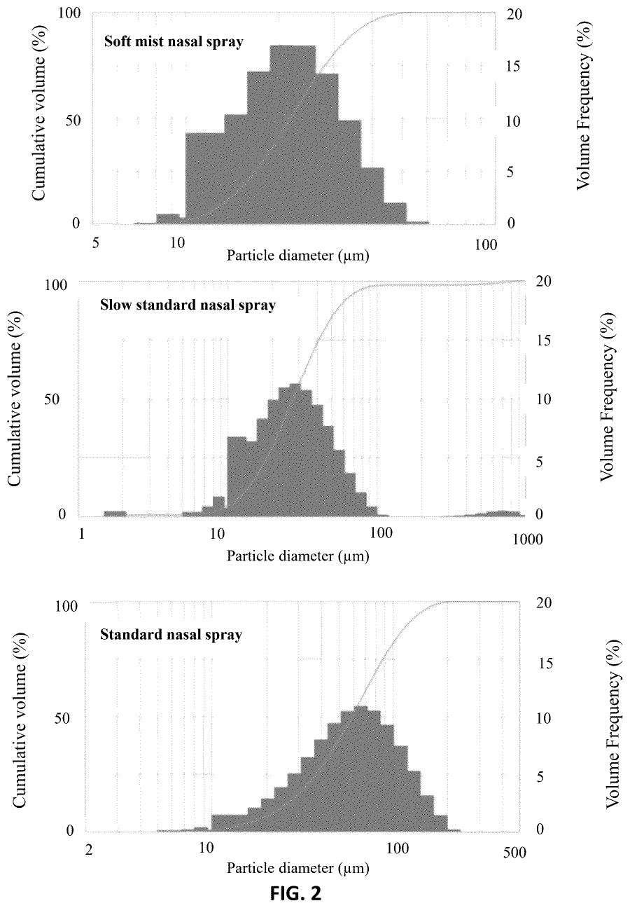

A theophylline liquid formulation was actuated with three spray devices: the soft mist nasal spray device, the slow standard nasal spray device, and the standard nasal spray device. The data the actuations are shown in FIG. 2. The left Y-axis shows the cumulative volume (%) of each spray and is shown by the sigmoidal line. The X-axis shows the particle diameter (μm) of the droplets. The right Y-axis shows the Volume Frequency (%) of the droplet sizes. The soft mist nasal spray had about 50% of the droplets with a particle diameter of less than 20 μm and substantially no particles over the size of 60 μm. The slow standard nasal spray had about 50% of the droplets with a particle diameter of less than 30 μm and the remaining 50% of the particles were about 30 μm to about 100 μm in size. The standard nasal spray had about 50% of the droplets with a particle diameter of less than about 60 μm and the remaining particles were about than about 60 μm to about 200 μm in size. As shown in FIG. 1 and FIG. 2, the small droplet size of the soft mist nasal spray focused the formulation to the olfactory region.

### Example 3: Treatment of Chemosensory Dysfunction in COVID-19 Patients

The Patient initially reported their sensory dysfunction as either loss of taste (i.e., flavor) and/or smell function. This subjective response was documented by objective psychophysical measurements of olfactory function administered to each patient by use of a forced-choice, three-stimuli, stepwise-staircase technique in a fixed, controlled design (1, 2). Efficacy of this technique and results of testing were previously documented in a double-blind clinical trial (2). Four odors were used; they were pyridine (dead-fish odor), nitrobenzene (bitter-almond odor), thiophene (petroleum-like odor) and amyl acetate (banana-oil odor). Detection thresholds (DT), and recognition thresholds (RT) values for each odor were determined as previously described (1, 2). Thresholds were converted into bottle units (BU) as previously described (2) and results reported as M of correct responses for each odor in each treatment group.

Theophylline was administered to a patient who had loss of taste and smell from COVID-19. A theophylline containing formulation was administered via two actuations to each naris from a standard nasal spray device once per day such that 80 μg in total of theophylline was administered. Prior to the initial administration of theophylline, a baseline smell test was performed. A follow-up smell test was performed 3 months after the initial administration. No side effects were reported. The results are shown in Table 2. The patient's detection threshold (DT) and recognition threshold (RT) for pyridine (Pyr), nitrobenzene (NO2B), thiophene (Thio) and amyl acetate (AA) was determined before treatment (pre) and after treatment (3M). In all four odorant tests, the patient's detection threshold and recognition threshold improved. An improvement of 1 bottle unit means that a patient could detect and/or recognize an odorant that was 10 times lower in concentration, when compared to baseline. For example, the recognition threshold (RT) of AA improved by 8 bottle units, also equivalent to an improvement of 8 orders of magnitude. In this case, the patient could detect a concentration of AA that was 10⁸ lower in concentration. Bottle unit numbers that are 5 or lower are considered to be in the range of normal.

TABLE 2
Results of Taste and Smell Testing on patient who had COVID-19

| | | DT | DT | DT | DT | RT | RT | RT | RT |
|---|---|---|---|---|---|---|---|---|---|
| | odor | Pyr | NO2B | Thio | AA | Pyr | NO2B | Thio | AA |
| Patient | Pre | 11 | 11 | 10 | 11 | 11 | 12 | 10 | 12 |
| | treatment | | | | | | | | |
| Patient | 3 months | 7 | 9 | 4 | 4 | 7 | 9 | 4 | 4 |

DT = Detection Threshold;
RT = Recognition Threshold

### Example 4: Theophylline Spray Formulation

Table 3 shows two example formulations for nasally administered theophylline

TABLE 3
Theophylline formulations

| | 1.75 mg/mL | 6.25 mg/mL |
|---|---|---|
| Ingredients | Quantity per mL | |
| Theophylline USP | 1.75 mg | 6.25 mg |

| | |
|---|---|
| Carboxymethylcellulose (CMC) | 2.83 mg |
| Citric acid | 0.30 mg |
| Sodium Hydroxide 1% solution | Quantum satis (Q.S) to target pH |
| Water for injection | Q.S to 1 mL |

### Example 5: Treatment of Phantosmia in a Subject

A subject is admitted to a clinic for phantosmia and phantogeusia. The subject is prescribed a thioridazine composition in a nasal spray device described herein. The subject administers the nasal spray in an effective dose 3× daily in each naris. The nasal spray is characterized by a spray containing a D₅₀ of about 15 μm to about 24 μm, a D₉₀ of from about 30 μm to about 50 μm and where less than about 3% of the droplets in the plume of a spray have a size of less than about 10 μm. The subject has reduced symptoms of the phantosmia and phantogeusia after administration of the thioridazine.

### Example 6: Soft Mist Nasal Spray Properties

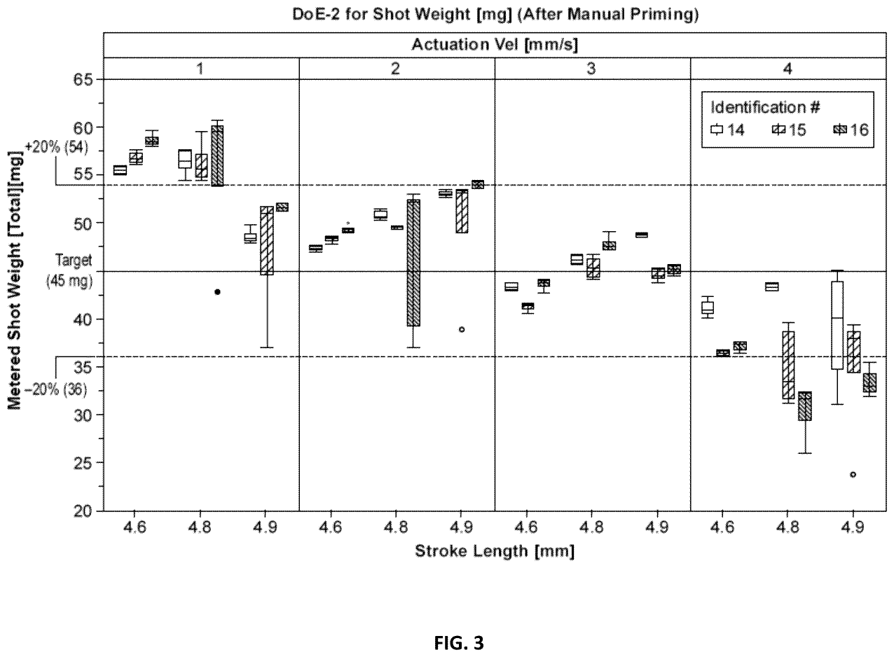

Soft mist nasal spray devices were tested to determine the spray characteristics of the soft mist nasal sprays upon actuation. 3 or 6 devices were tested using an automated system. The stroke lengths of actuation of the nasal spray device were 4.6 mm, 4.8 mm and 4.9 mm. The actuator stroke (AS) acceleration was about 500 millimeters/second 2 (mm/s²), The AS velocities were 1, 2, 3, or 4 millimeters/second (mm/s). The average hold time (i.e. average amount of time depressing the spray device) was 684 milliseconds (ms). Each experiment was tested with 3 or 6 dosing shots per AS velocity for each device. The devices were primed with several actuations before testing. The composition was tested in a formulation comprising 2.8 mg/ml theophylline, preservatives (benzalkonium chloride and phenylethyl alcohol), and saline. The spray device had 48 nozzle pores at a size of 4 μm per nozzle pore and a 20° cone angle. The box and whisker graphs of FIGS. 3-6, and 8-13 are described as follows. The box is the range of 25th to 75th percentile. The line in the box is the median (50th percentile). The whiskers are the max and minimum—unless deemed to be outliers, which are shown with an asterisk. No whiskers indicate that the lower quartile is equal to the minimum and the upper quartile is equal to the maximum.

The metered shot weight (amount released in mg) is shown in FIG. 3. FIG. 3 depicts a box and whisker graph showing the metered shot weight (amount released in milligrams (mg)) from soft mist pump devices on the Y-axis, at different actuation velocities (1, 2, 3, and 4 millimeters per second (mm/s)) on the upper X-axis, and 4.6 mm, 4.8 mm, and 4.9 mm stroke lengths shown on the lower X-axis. Actuation velocities of 2 mm/s and 3 mm/s were within about 20% of the target shot weight of 45 mg.

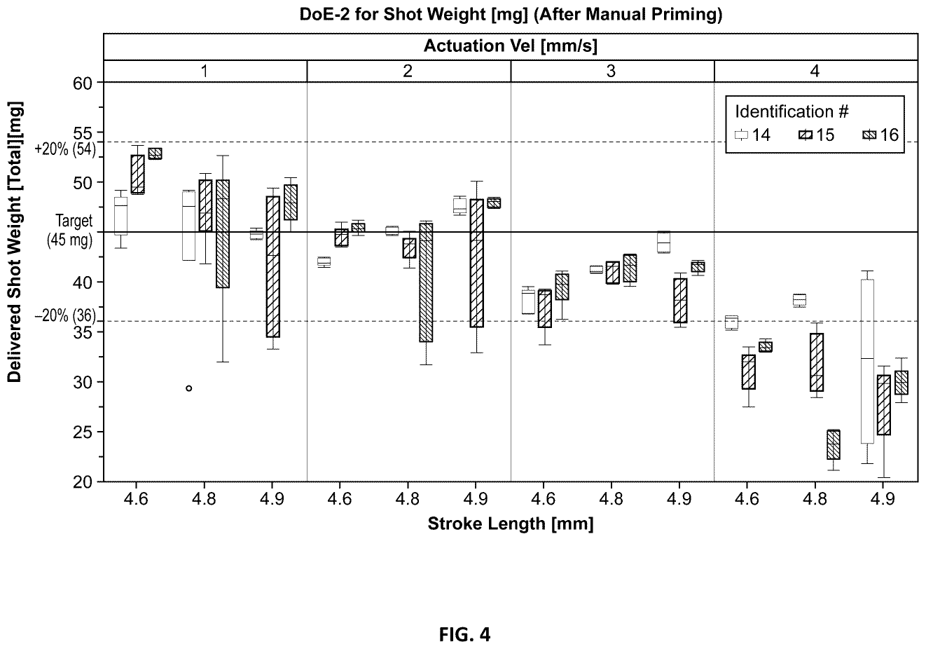

The delivered shot weight (amount delivered in mg) is shown in FIG. 4. FIG. 4 depicts a box and whisker graph showing the delivered shot weight (amount delivered in milligrams (mg)) from soft mist pump devices on the Y-axis, at different actuation velocities (1, 2, 3, and 4 millimeters per second (mm/s)) on the upper X-axis, and 4.6 mm, 4.8 mm, and 4.9 mm stroke lengths shown on the lower X-axis. Actuation velocities of 1 mm/s, 2 mm/s, and 3 mm/s were within about 20% of the target shot weight of 45 mg.

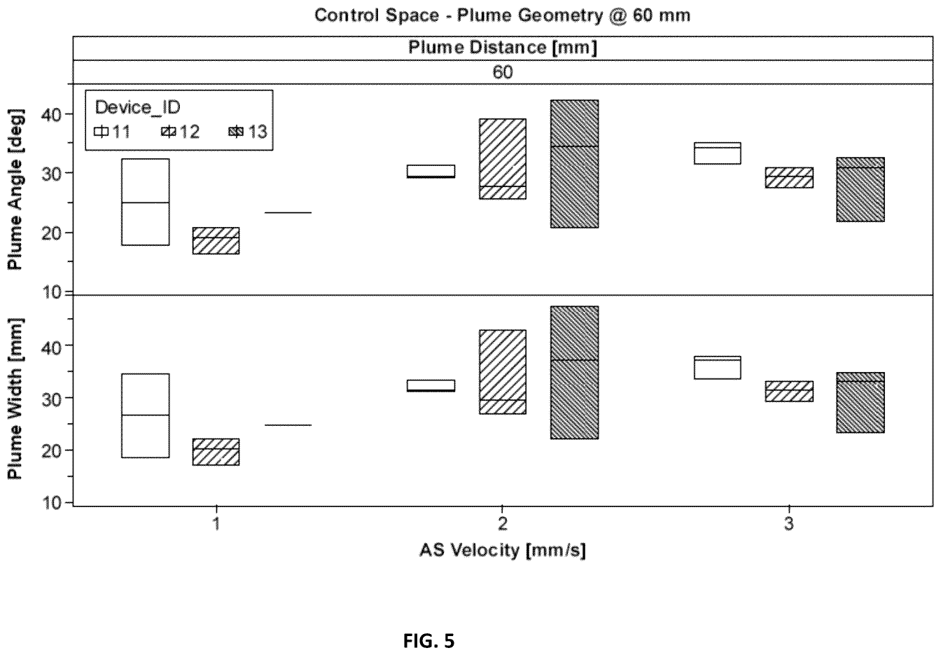

The spray plume geometry at a 60 mm pattern distance is shown in FIG. 5. FIG. 5 depicts a box and whisker graph showing the plume geometry performance (Plume Angle (degree) and Plume Width (mm)) from soft mist pump devices on the Y-axis, at different actuation velocities (1, 2, 3 millimeters per second (mm/s)) on the X-axis at a 4.8 mm stroke length. The plume angle was about 15 degrees to about 40 degrees. The plume width was about 15 mm to about 45 mm. Several spray patterns were unable to be determined, such as 1 replicate for 1 mm/sec, for device #11, and 2 replicates for 1 mm/sec for device #13.

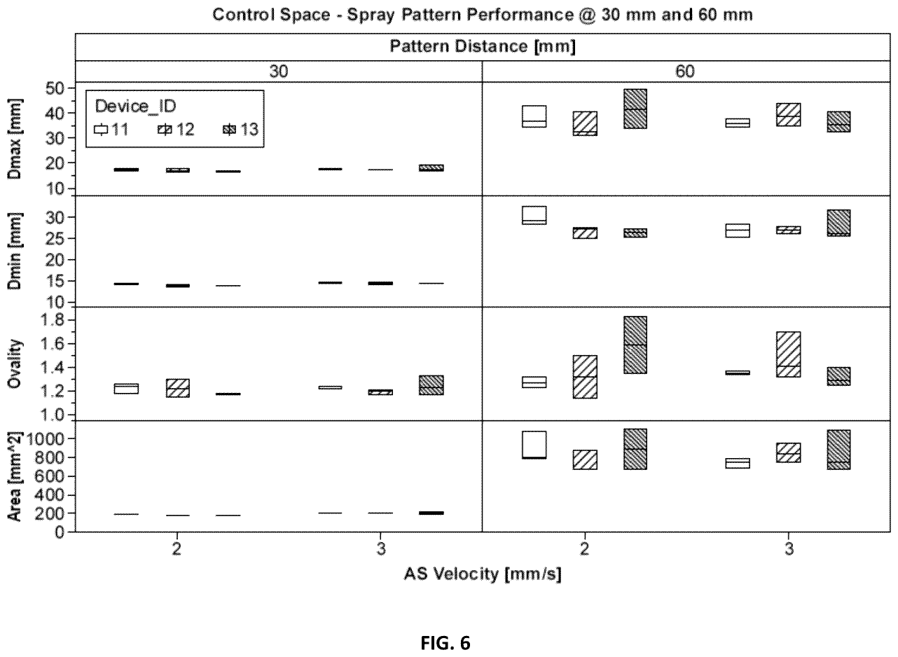

The spray pattern performance at 30 mm and 60 mm is shown in FIG. 6. FIG. 6 depicts a box and whisker graph showing the spray pattern characteristics of Dmax (mm), Dmin (mm), ovality and area (mm\hat{ }2) on the Y-axis, at different actuation velocities (2 mm/s and 3 mm/s) on the lower X-axis at 30 mm and 60 mm pattern distances shown on the upper X-axis at a 4.8 mm stroke length. For a spray distance at 30 mm, the Dmax (max diameter) was 15-20 mm, the Dmin (minimum diameter) was about 14 mm, the ovality was about 1.1-1.3, and the area was about 180-200 mm\hat{ }2. For a spray distance at 60 mm, the Dmax (max diameter) was about 30-50 mm, the Dmin (minimum diameter) was about 20-30 mm, the ovality was about 1.1-1.8, and the area was about 600-1000 mm\hat{ }2. At 60 mm, a replicate at 2 mm/sec of device #13 did not result in a quantifiable pattern.

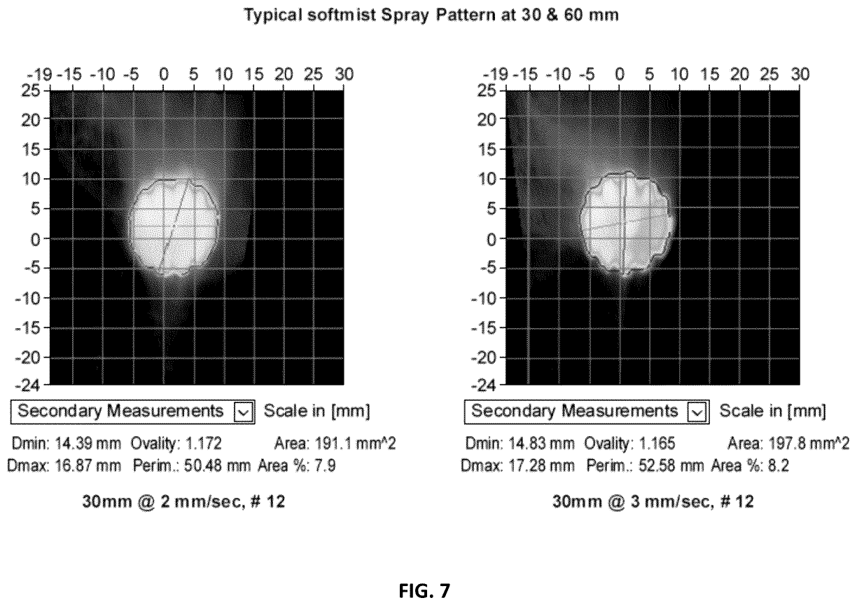

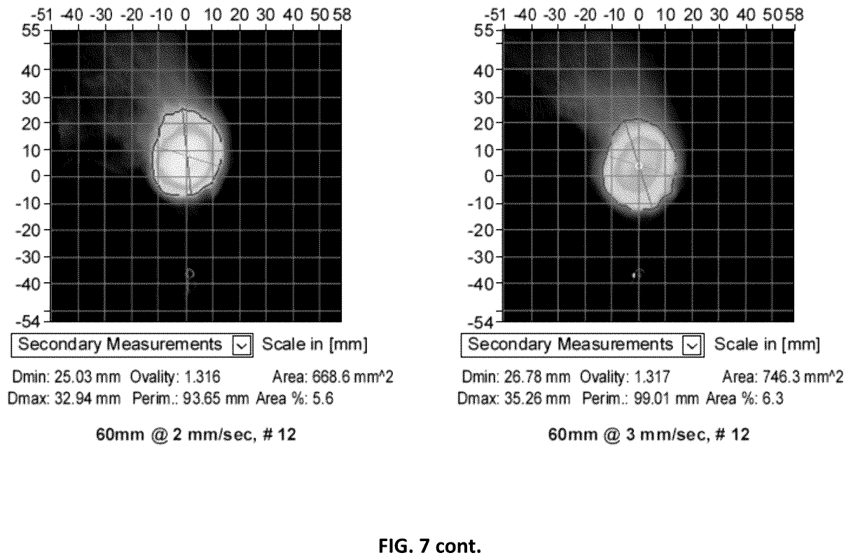

The spray patterns at 30 mm and 60 mm pattern distances are shown in FIG. 7. FIG. 7 depicts a images showing the spray patterns at 30 mm and 60 mm pattern distances from a soft mist pump device (device 12) at different actuation velocities (2 and 3 millimeters per second (mm/s)) and at a 4.8 mm stroke length. In some instances, spray pattern parameters at 60 mm could not be calculated due to the dissipation of spray produced at 60 mm. The images were captured by a non-impact laser method on Proveris Sprayview equipment. In the non-impact laser method, a sheet of laser light is projected and an image is produced of the concentration of droplets across the laser light plane.

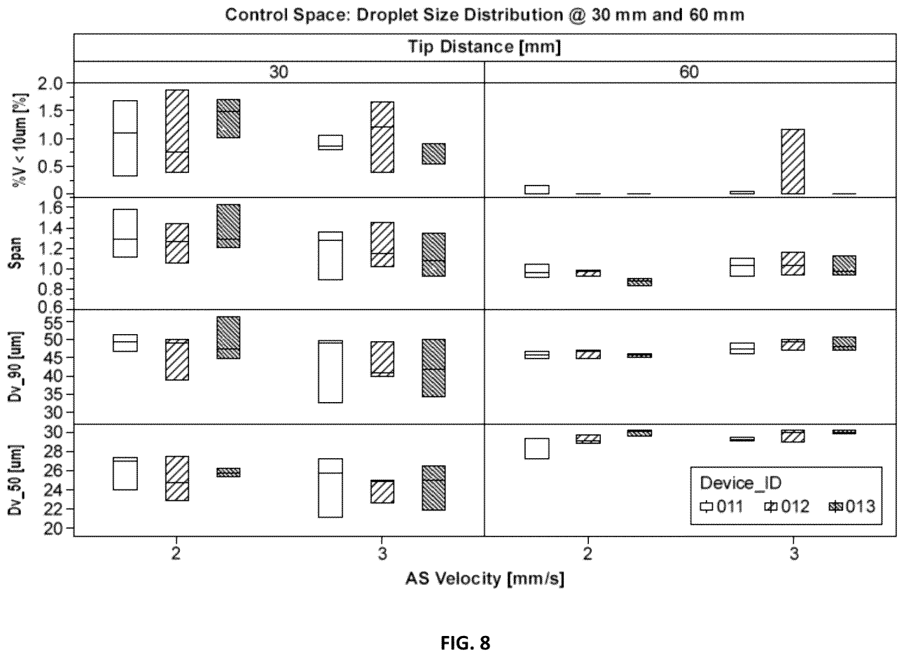

The droplet size distributions at 30 mm and 60 mm pattern distances are shown in FIG. 8. FIG. 8 depicts a box and whisker graph showing the droplet size distribution (% volume<10 μm; span; D90 value (μm); and D50 value (μm)) on the Y-axis at different actuation velocities (2 mm/s and 3 mm/s) on the lower X-axis at 30 mm and 60 mm pattern distances shown on the upper X-axis at a 4.8 mm stroke length. For spray devices at 30 mm, the % volume<10 μm was about 0.25% to about 2%, the span was about 0.8 to about 1.6, the D₉₀ value was about 30-55 μm; and the D₅₀ value was about 21-28 μm. For spray devices at 60 mm, the % volume<10 μm was less than about 1%, the span was about 0.8 to about 1.2, the D₉₀ value was about 40-50 μm; and the D₅₀ value was about 26-30 μm.

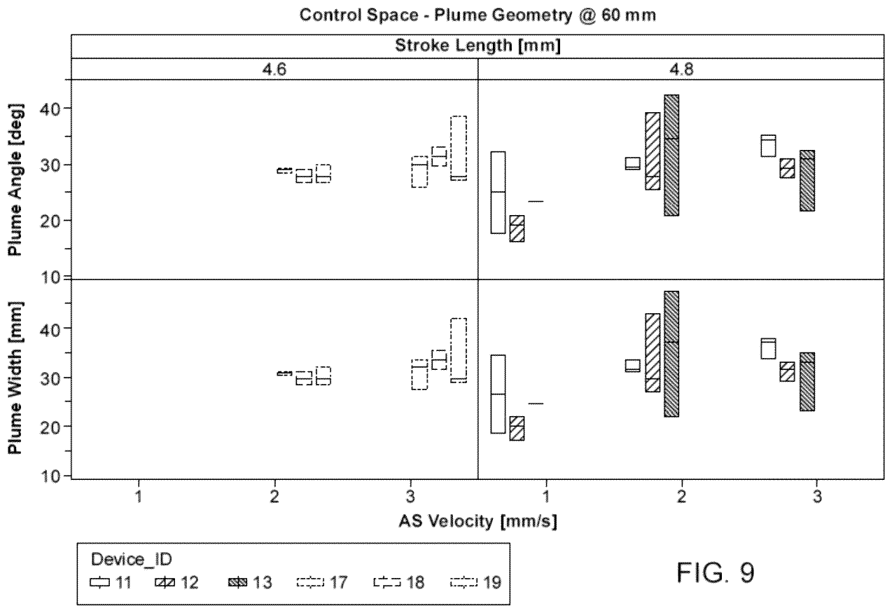

The plume geometry at a pattern distance of 60 mm is shown in FIG. 9. FIG. 9 depicts a box and whisker graph showing the plume geometry performance (Plume Angle (degree) and Plume Width (mm)) on the Y-axis, at different actuation velocities (1, 2, 3 millimeters per second (mm/s)) on the lower X-axis, at a 4.6 mm and 4.8 mm stroke length on the upper X-axis. At a stroke length of 4.6 mm, the plume angle was about 25-40 degrees and the plume width was about 25-40 mm. At a stroke length of 4.8 mm, the plume angle was about 15-40 degrees and the plume width was about 15-40 mm. In some instances, a spray was not detected at the 1 mm/sec velocity.

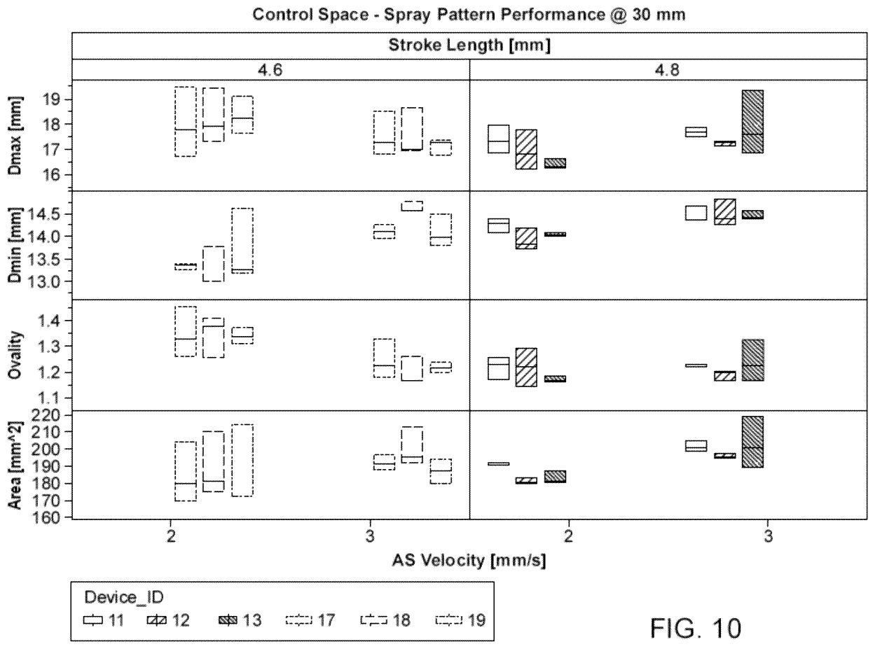

The spray pattern performance at a pattern distance of 30 mm is shown in FIG. 10. FIG. 10 depicts a box and whisker graph showing the spray pattern (Dmax (mm), Dmin (mm), ovality and area (mm\hat{ }2)) on the Y-axis, at different actuation velocities (2 and 3 mm/s) on the lower X-axis, at 4.6 mm and 4.8 mm stroke lengths of the upper X-axis. At a stoke length of 4.6 mm, the Dmax was about 16-20 mm, the Dmin was about 13-14.5 mm, the ovality was about 1.1-1.4 and the area was about 170-210 mm\hat{ }2. At a stoke length of 4.8 mm, the Dmax was about 16-20 mm, the Dmin was about 13.5-14.75 mm, the ovality was about 1.1-1.3 and the area was about 170-210 mm\hat{ }2.

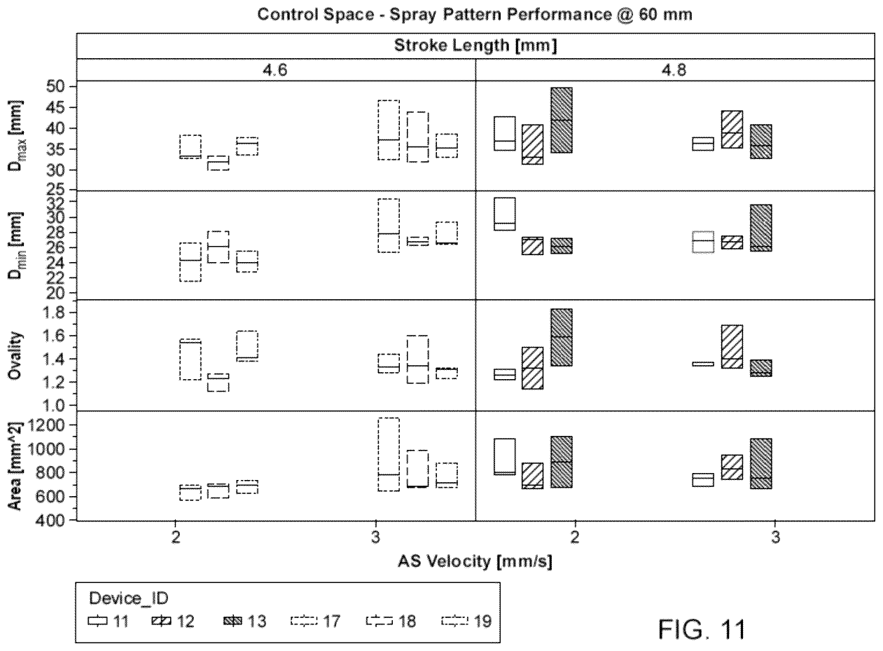

The spray pattern performance at a pattern distance of 60 mm is shown in FIG. 11. FIG. 11 depicts a box and whisker graph showing the spray pattern (Dmax (mm), Dmin (mm), ovality and area (mm\hat{ }2)) on the Y-axis, at different actuation velocities (2 and 3 mm/s) on the lower X-axis, at 4.6 mm and 4.8 mm stroke lengths of the upper X-axis. At a stoke length of 4.6 mm, the Dmax was about 30-45 mm, the Dmin was about 21-32 mm, the ovality was about 1.1-1.6 and the area was about 500-1200 mm\hat{ }2. At a stoke length of 4.8 mm, the Dmax was about 30-50 mm, the Dmin was about 24-32 mm, the ovality was about 1.1-1.8 and the area was about 600-1000 mm\hat{ }2. In some instances, spray pattern parameters at 60 mm could not be calculated due dissipation of the spray at the 60 mm distance.

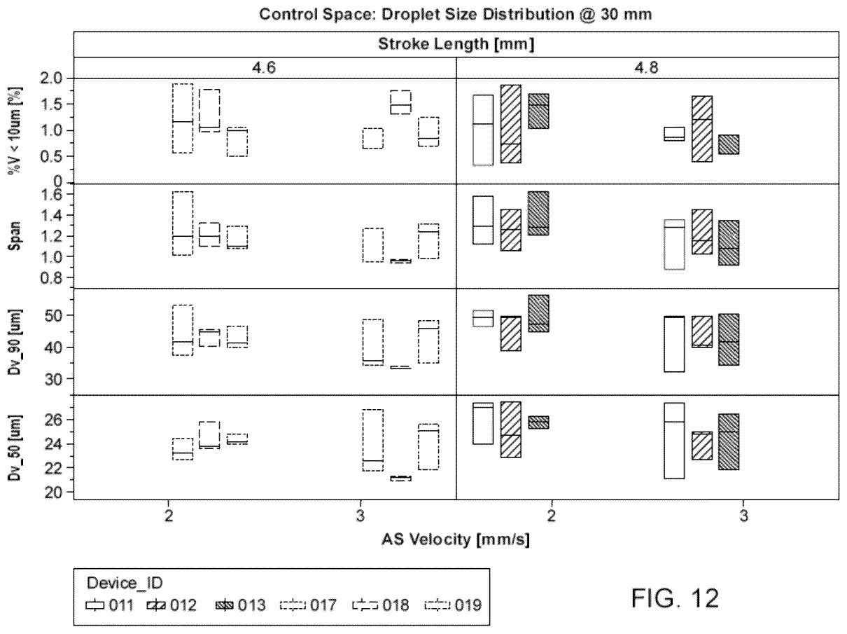

The droplet size distribution at a spray distance of 30 mm is shown in FIG. 12. FIG. 12 depicts a box and whisker graph showing the droplet size distribution (% volume<10 μm; span; D90 value (μm); and D50 value (μm)) on the Y-axis, at different actuation velocities (2 and 3 mm/s) on the lower X-axis, at 4.6 mm and 4.8 mm stroke lengths on the upper X-axis. At the 4.6 mm stroke length, the % volume<10 μm was about 0.5-2%, the span was about 0.8 to about 1.6, the D₉₀ value was about 35-52 μm; and the D₅₀ value was about 21-26 μm. At the 4.8 mm stroke length, the % volume<10 μm was about 0.25-1.75%, the span was about 0.8 to about 1.6, the D₉₀ value was about 30-55 μm; and the D₅₀ value was about 21-27 μm.

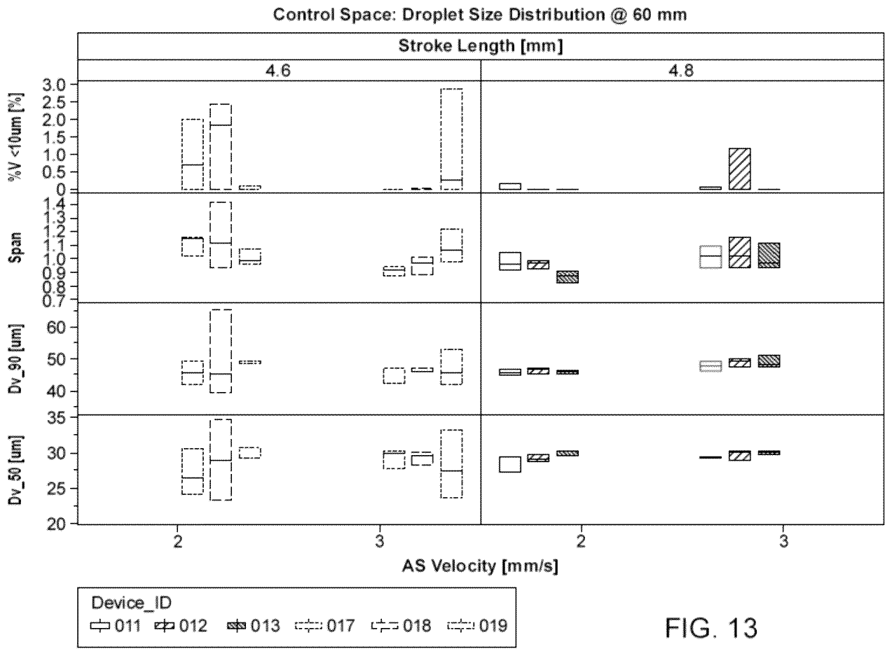

The droplet size distribution at a spray distance of 60 mm is shown in FIG. 13. FIG. 13 depicts a box and whisker graph showing the droplet size distribution (% volume<10 μm; span; D90 value (μm); and D50 value (μm)) on the Y-axis, at different actuation velocities (2 and 3 min/s) on the lower X-axis, at 4.6 mm and 4.8 mm stroke lengths on the upper X-axis. At the 4.6 mm stroke length, the 00 volume<10 μm was less than 2.5%, the span was about 0.8 to about 1.4, the D₉₀ value was about 36-63 μm; and the D₅₀ value was about 23-34 μm. At the 4.8 mm stroke length, the 00 volume<10 μm was less than about 100, the span was about 0.8 to about 1.1, the D₉₀ value was about 40-50 μm; and the D₅₀ value was about 25-30 μm.

A summary of the droplet size distributions are presented in Table 4. Table 4 shows the droplet size distributions according to: pattern distance or tip distance (30 mm or 60 mm); actuator stroke velocities (2 mm/s and 3 mm/s); and stroke lengths (4.6 mm and 4.8 mm). The mean, standard deviation and coefficient of variation (CV) of the of the D10 value (μm), the D₅₀ value (μm), the D₉₀ value (μm), the percent volume<10 μm, the percent volume<5 μm, and the span are shown for the replicates of the tested devices.

TABLE 4
Summary of droplet size distributions by soft mist spray device

| | | Tip Distance [mm] | | | | | | | |
|---|---|---|---|---|---|---|---|---|---|
| | | 30 | | | | 60 | | | |
| | | AS Velocity [mm/s] | | | | AS Velocity [mm/s] | | | |
| | | 2 | | 3 | | 2 | | 3 | |
| | | Stroke Length [mm] | | | | Stroke Length [mm] | | | |
| Parameter | | 4.6 | 4.8 | 4.6 | 4.8 | 4.6 | 4.8 | 4.6 | 4.8 |
| D10 [um] | Mean | 14.092 | 14.376 | 13.95 | 14.318 | 16.702 | 18.523 | 17.821 | 17.913 |
| | Std | 0.472 | 0.767 | 0.624 | 0.587 | 2.248 | 1.065 | 2.23 | 0.88 |
| | Dev | | | | | | | | |
| | CV | 3.35 | 5.339 | 4.475 | 4.101 | 13.459 | 5.751 | 12.516 | 4.915 |
| D50 [um] | Mean | 24.087 | 25.686 | 23.057 | 24.457 | 28.731 | 29.343 | 28.874 | 29.73 |
| | Std | 0.913 | 1.579 | 2.23 | 2.103 | 3.553 | 0.889 | 2.599 | 0.494 |
| | Dev | | | | | | | | |
| | CV | 3.79 | 6.148 | 9.672 | 8.597 | 12.365 | 3.028 | 9 | 1.663 |
| D90[um] | Mean | 43.423 | 48.267 | 38.759 | 43.114 | 48.179 | 45.96 | 46.176 | 48.431 |
| | Std | 4.682 | 4.865 | 6.758 | 6.981 | 7.387 | 0.844 | 3.22 | 1.534 |
| | Dev | | | | | | | | |
| | CV | 10.781 | 10.08 | 17.437 | 16.192 | 15.333 | 1.836 | 6.974 | 3.167 |
| % V < 10 um | Mean | 1.116 | 1.153 | 1.126 | 0.936 | 0.789 | 0.017 | 0.352 | 0.138 |
| [%] | Std | 0.463 | 0.577 | 0.362 | 0.365 | 1.023 | 0.051 | 0.946 | 0.384 |
| | Dev | | | | | | | | |
| | CV | 41.452 | 50.023 | 32.183 | 39.056 | 129.663 | 293.735 | 268.709 | 278.142 |
| % V < 5 um | Mean | 0 | 0 | 0 | 0 | 0.058 | 0 | 0 | 0 |
| [%] | Std | 0 | 0 | 0 | 0 | 0.174 | 0 | 0 | 0 |
| | Dev | | | | | | | | |
| | CV | . | . | . | . | 300 | . | . | . |
| Span | Mean | 1.216 | 1.32 | 1.065 | 1.166 | 1.095 | 0.936 | 0.988 | 1.024 |
| | Std | 0.185 | 0.195 | 0.157 | 0.202 | 0.148 | 0.064 | 0.107 | 0.086 |
| | Dev | | | | | | | | |
| | CV | 15.224 | 14.772 | 14.739 | 17.315 | 13.555 | 6.884 | 10.875 | 8.415 |

### Example 7: Treatment of Parkinson's Disease Chemosensory Dysfunction

An adult human male subject was treated for chemosensory dysfunction associated with Parkinson's disease. For two years prior to treatment the subject did not have the ability to taste or smell. The subject was administered about 0.4 ml per day of a liquid composition comprising theophylline at a concentration of 3.1 mg/ml in a nasal spray device described herein. The subject administered the nasal spray twice daily in each naris. The subject regained their ability to taste and smell after using the spray device for 7 days.

### Example 8: Non-aqueous Nasal Formulations of Theophylline

Two non-aqueous theophylline formulations were developed and are shown in Table 5 and Table 6. In some instances, the drug concentration (e.g., a PDE inhibitor) in the formulation can be as high as 12 mg/mL. The formulations can be used with the nasal spray device described herein.

TABLE 5
Formulation A: 5.7 mg/mL theophylline

| Ingredient | Function | grams |
|---|---|---|
| Theophylline | Active ingredient | 0.57 |
| DMSO | Solvent/vehicle | 5 |
| Oleyl Alcohol | Solvent, permeation | 5 |
| | enhancer | |
| DEGEE (Transcutol P) | Solvent, permeation | 25 |
| | enhancer | |
| Dipropylene Glycol | Solvent, permeation | 5 |
| | enhancer | |
| PEG 400 | Solvent/vehicle | 10 |
| Propylene Glycol | Solvent, permeation | 50 |
| | enhancer | |
| Hydroxypropyl | Solution thickener | 0.04 |
| Cellulose | | |
| Total | | 100.61 |

TABLE 6
Formulation B: 11 mg/mL theophylline

| Ingredient | grams |
|---|---|
| Theophylline | 1.11 |
| DMSO | 5 |
| Oleyl Alcohol | 5 |
| DEGEE (Transcutol P) | 25 |
| Dipropylene Glycol | 5 |
| PEG 400 | 10 |
| Propylene Glycol | 50 |
| Hydroxypropyl Cellulose | 0.4 |
| Total | 101.51 |

While preferred embodiments of the present disclosure have been shown and described herein, such embodiments are provided by way of example only. It should be understood that various alternatives to the embodiments of the disclosure described herein may be employed in practicing the disclosure.

What is claimed is:

1. A liquid pharmaceutical composition, wherein the liquid pharmaceutical composition comprising:
about 1.75 or about 3.1 mg/mL theophylline or a pharmaceutically acceptable salt thereof;
0.01% to about 0.5% wt/wt of benzalkonium chloride;
citric acid;
sodium chloride; and
water.

2. The liquid pharmaceutical composition of claim 1, wherein the liquid pharmaceutical composition comprises about 0.02% wt/wt of benzalkonium chloride.

3. The liquid pharmaceutical composition of claim 1, wherein the liquid pharmaceutical composition is a pH of 6.5.

4. The liquid pharmaceutical composition of claim 1, wherein the liquid pharmaceutical composition comprises sodium hydroxide and/or hydrochloric acid in an amount to adjust the pH of the formulation to 6.5.

5. The liquid pharmaceutical composition of claim 1, wherein the liquid pharmaceutical composition comprises 0.10% to about 0.3% wt/wt of phenylethyl alcohol.

6. The liquid pharmaceutical composition of claim 1, wherein the liquid pharmaceutical composition comprises about 0.2% wt/wt of phenylethyl alcohol.

7. A liquid pharmaceutical composition, the liquid pharmaceutical composition comprising:
about 1.75 to 6.25 mg/mL of theophylline or a pharmaceutically acceptable salt thereof;
about 0.01% to about 0.3% wt/wt of one or more preservatives;
a buffering agent; and
water.

8. The liquid pharmaceutical composition of claim 7, wherein the liquid pharmaceutical composition comprises about 1.75 mg/mL or about 3.1 mg/mL theophylline or a pharmaceutically acceptable salt thereof.

9. The liquid pharmaceutical composition of claim 7, wherein the one or more preservatives comprises benzalkonium chloride.

10. The liquid pharmaceutical composition of claim 7, wherein the one or more preservatives comprises benzalkonium chloride and phenylethyl alcohol.

11. The liquid pharmaceutical composition of claim 7, wherein the total amount of the one or more preservatives is from 0.02% wt/wt to about 0.3% wt/wt.

12. The liquid pharmaceutical composition of claim 7, wherein the liquid pharmaceutical composition comprises citric acid and/or sodium chloride.

13. The liquid pharmaceutical composition of claim 7, wherein the liquid pharmaceutical composition comprises sodium hydroxide and/or hydrochloric acid.

14. A nasal spray device comprising the liquid pharmaceutical composition of claim 1.

15. The nasal spray device of claim 14, wherein the nasal spray device is configured to deliver a dosage unit in a plume upon actuation.

16. The nasal spray device of claim 15, wherein the dosage unit comprises 100 μg to 300 μg theophylline.

17. The nasal spray device of claim 15, wherein the plume has a droplet size distribution characterized by a D₉₀ of about 35 μm to about 45 μm wherein about 90% of the droplets in the plume have a size less than the D₉₀; wherein the plume has a droplet size distribution characterized by a by a D₅₀ of about 20 μm to about 30 μm, wherein about 50% of the droplets in the plume have a size less than the D₅₀; and/or wherein the plume has a droplet size distribution characterized by a D₁₀ of about 8 μm to about 15 μm, wherein about 10% of the droplets in the plume have a size less than the D₁₀.

18. A kit comprising the nasal spray device of claim 15 and instructions for use.

19. A method of treating hyposmia, the method comprising nasally administering the liquid pharmaceutical composition of claim 1.

20. The method of claim 19, comprising nasally administering the liquid pharmaceutical composition in unit dose form in each naris twice per day so that the daily dose comprises 500 to 1200 μg theophylline.
